SEQUENCE LISTING (7 sequences)

SEQ ID NO: 1 - DNA, 16 bp, Artificial Sequence
  antisense Syk kinase PNA sequence
attttttgacatggga

SEQ ID NO: 2 - DNA, 16 bp, Artificial Sequence
  antisense ZAP-70 kinase PNA sequence
gtttgcgctcggcctc

SEQ ID NO: 3 - DNA, 23 bp, Artificial Sequence
  PCR primer for gamma IFN sense
tgtaagcccccagaaacagaaag

SEQ ID NO: 4 - DNA, 21 bp, Artificial Sequence
  PCR primer for gamma IFN antisense
ttgcccatcaagaaacagcag

SEQ ID NO: 5 - DNA, 24 bp, Artificial Sequence
  PCR primer for IL-4 sense
tcactcttcactcttttcttcccc

SEQ ID NO: 6 - DNA, 22 bp, Artificial Sequence
  PCR primer for IL-4 antisense
tcttcccactttgctgttcctc

SEQ ID NO: 7 - DNA, 27 bp, Homo sapiens
ggaggaaaaactgtttcatacagaagg

(19) United States
(12) Patent Application Publication
(10) Pub. No.: US 2003/0103938 A1
(43) Pub. Date: Jun. 5, 2003

(54) Pharmaceutical compositions for preventing or treating Th1 and Th2 cell related diseases by modulating the Th1/Th2 ratio

(75) Inventors: Tan Jinquan, Frederiksberg C (DK); Lars Kaergaard Poulsen, Vedbaek (DK)

Correspondence Address: DARBY & DARBY P.C., P. O. BOX 5257, NEW YORK, NY, 10150-5257, US

(73) Assignee: ALK-ABELLO A/S

(21) Appl. No.: 10/143,528

(22) Filed: May 9, 2002

Related U.S. Application Data
(60) Provisional application No. 60/289,711, filed on May 9, 2001.

(51) Int. Cl. A61K 38/19

(52) U.S. Cl. 424/85.2; 514/12

(57) ABSTRACT

Pharmaceutical composition for preventing or treating a Th1 cell- or Th2-cell-related disease in a human or an animal by modulating the Th1/Th2 ratio comprising an active substance consisting of (i) IL-4 and SDF-1α, or IL-2 and SDF-1α, respectively, as well as modulators thereof, (ii) an IL-4 stimulating adjuvant and SDF-1α, or an IL-2 stimulating adjuvant and SDF-1α, respectively, (iii) a modulator of the tyrosine kinases Syk or ZAP-70, or (iv) a modulator of the nuclear factors of activated T cells NFAT1 or NFAT2.

### FIELD OF THE INVENTION

The present invention relates to a pharmaceutical composition for preventing or treating a Th1 cell-related disease in a human or an animal by reducing the Th1/Th2 ratio.

Similarly, the present invention relates to pharmaceutical composition for preventing or treating a Th2 cell-related disease in a human or an animal by increasing the Th1/Th2 ratio.

### BACKGROUND OF THE INVENTION

The CD4⁺ T helper cells differentiate into either Th1 or Th2 T cells depending upon antigen stimulation and cytokine environment. So far, it is not very clear whether instructive or/and selective differentiation may contribute to Th cell formation in vivo. T helper cells cells first activated by antigen in the presence of IL-12 develop predominantly into Th1 cells, whereas those activated in the presence of IL-4 develop predominantly into Th2 cells (Coffman et al., 1999). It is believed that progenitor Th cells require individual cellular divisions before becoming competent to synthesize cytokines indicative of either the Th1 or Th2 pathway (Bird et al., 1998). The Syk-family including Syk and ZAP-70 tyrosine kinases are essential for lymphocyte development and activation. Despite clear structural resemblance between Syk and ZAP-70, there is growing evidence that these two kinases are differentially regulated, both in terms of expression and activity (Chu et al., 1998). Th1 and Th2 cell phenotypes are different from each other in early activation signal transduction pathways, especially in the role of TCR related protein tyrosine kinases (Tamura et al., 1995). TCR and its downstream protein tyrosine kinases such as Fyn, p56(Ick), and ZAP-70 are involved in the development and differentiation of Th1 and Th2 cells (Swith et al., 1998; Faith et al., 1997; al-Ramadi et al., 1996; Deckert et al., 1998). Cbl is an adaptor protein that functions as a negative regulator of many signaling pathways such as Syk family that start from receptors at the cell surface (Meng et al., 1999; Ota et al., 1997). Cbl-b, a homologue of Cbl, has a positive role in T-cell signaling via a direct interaction with the upstream kinase ZAP-70 (Zhang et al., 1999). CXCR4, the only known receptor for SDF-1α, can cross-talk with TCR to affect anti-CD3-stimulated phosphorylation of critical downstream substrates of TCR signaling including ZAP-70 (Peacock et al., 1999). Despite significant progress, the signaling pathways involved in the development and dynamics of Th1 and Th2 cells remain unclear.

Jourdan et al., 1998 discloses the finding that IL-4 specifically enhances cell surface expression of CXCR4 in Th2 cells and also in Th1 cells. The CXCR4 ligand SDF-1α activates the p42 MAP-kinase ERK-2. Such activation renders T cells susceptible to HIV infection and promotes SDF-1α induced migration of the cells. In the study, the activity of p42 MAP-kinase was measured in order to examine whether the IL-4 induced CXCR4 was functional. In addition, the activity of ZAP-70 tyrosine kinase was measured as a measure of the global level of signalling proteins.

Th2 responses are enhanced in NFAT1 knock-out mice (Hodge et al. 1996; Viola et al. 1998) and Th2 responses are impaired in NFAT2 knock-out mice (Ranger et al. 1998; Yoshida et al. 1998).

WO 00/24245 discloses the technical teaching that NFATp (NFAT1) and NFAT4 in combination act as selective repressors of Th2 cells. The document describes a) mice deficient in both NFATp and NFAT4 that exhibit a phenotype characteristic of increased Th2 cell activity, b) methods of identifying modulators of Th2 cell activity, using either cells deficient in both NFAT p and NFAT4, mice deficient in both NFATp and NFAT4 or indicator compositions containing both NFATp and NFAT4, c) methods of regulating Th2 cell activity using agents that modulate the activity of NFATp and NFAT4, and d) methods of diagnosing disorders associated with aberrant Th2 cell activity by assessing changes in NFATp and/or NFAT4 expression.

U.S. Pat. No. 5,958,671 discloses methods of modulating production of a Th2-associated cytokine, in particular interleukin-4 (IL-4), by modulating the activity of c-Maf and methods of modulating development of Th1 and Th2 subsets in a subject using agents that modulate transcription factor activity. In particular, the document describes a method of stimulating IL-4 production by using a first agent to stimulate the expression or activity of c-Maf in combination with a second agent that stimulates the expression or activity of any member of the NFAT family, preferably NFATp. Also, the document describes a method of inhibiting IL4 production by using a first agent to inhibit the expression or activity of a maf family protein in combination with a second agent that inhibits the expression or activity of any member of the NFAT family.

### OBJECTIVE OF THE INVENTION

The objective of the invention is to provide therapeutic substances for treating Th1 and Th2 cell-related diseases.

### SUMMARY OF THE INVENTION

The objective of the invention is obtained with the first aspect of the invention, which relates to a pharmaceutical composition for preventing or treating a Th1 cell-related disease in a human or an animal by reducing the Th1/Th2 ratio comprising an active substance selected from the group consisting of a) IL-4 and SDF-1α, b) a stimulant of IL-4 and a stimulant of SDF-1α, c) an antagonist to IL-2 and an antagonist to SDF-1α, d) an inhibitor of Syk or NFAT1, e) a stimulant of ZAP-70 or NFAT2, f) an IL-4 stimulating adjuvant and SDF-1α, g) a functional derivative, analogue or part of any of the substances a)-f) or h) a combination of any of the substances a)-g).

Furthermore, the object of the invention is obtained with the second aspect of the invention, which relates to pharmaceutical composition for preventing or treating a Th2 cell-related disease in a human or an animal by increasing the Th1/Th2 ratio comprising an active substance selected from the group consisting of o) IL-2 and SDF-1α, p) a stimulant of IL-2 and a stimulant of SDF-1α, q) an antagonist to IL-4 and an antagonist to SDF-1α, r) an inhibitor of ZAP-70 or NFAT2, s) a stimulant of Syk or NFAT1, t) an IL-2 stimulating adjuvant and SDF-1α, u) a functional derivative, analogue or part of any of the substances o)-t) or v) a combination of any of the substances o)-u).

The present invention is based on the discovery of several steps in the mechanisms, which determine the differentiation of progenitor T cells into Th1 and Th2 cells, both at the extracellular and the intracellular level. Firstly, the present invention is based on the discovery that stimulation of a progenitor T cell with IL-2 or an IL-2 stimulating adjuvant in conjunction with SDF-1α causes the CD4⁺ T cell to develop into a Th1 cell. Secondly, it was found that stimulation of a progenitor T cell with IL-4 or an IL-4 stimulating adjuvant in conjunction with SDF-1α causes the CD4⁺ T cell to develop into a Th2 cell. It is believed that IL-2, IL-4, adjuvants and SDF-1α bind to their respective receptors on the surface of the progenitor T cells and trigger specific intracellular regulatory pathways leading to the said differentiation into either a Th1 or a Th2 cell.

Thirdly, the present invention is based on the discovery that the pathway leading to differentiation of a T cell into a Th1 cell involves the activation of the tyrosine kinase Syk as well as the transcription factor NFAT1, whereas the other NFAT transcription factors are not activated. Fourthly, it was found that the pathway leading to differentiation of a T cell into a Th2 cell involves the activation of the tyrosine kinase ZAP-70 as well as the transcription factor NFAT2, whereas the other NFAT transcription factors are not activated.

In view of the above mentioned findings as to the mechanisms of CD4⁺ T cell differentiation into Th1 and Th2 cells, the present invention is further based on the recognition that any substance capable of interfering with the function of any of substances found to be involved in the said T cell differentiation may be used as an active substance for preventing or treating diseases, which are mediated by either Th1 or Th2 cells. In particular, it is possible to treat a Th1 cell-related disease, i.e. a disease in which Th1 cells support the disease process, by either by inhibiting the differentiation of T cells into Th1 cells or by stimulating the differentiation of T cells into counteracting Th2 cells. Correspondingly, it is possible to treat a Th2 cell-related disease, i.e. a disease in which Th2 cells support the disease process, by either inhibiting the differentiation of T cells into Th2 cells, or by stimulating the differentiation of T cells into counteracting Th1 cells.

The present invention relates further to the following:

A pharmaceutical composition comprising an active substance selected from the group consisting of a) IL-4 and SDF-1α, b) a stimulant of IL-4 and a stimulant of SDF-1α, c) an antagonist to IL-2 and an antagonist to SDF-1α, d) an inhibitor of Syk or NFAT1, e) a stimulant of ZAP-70 or NFAT2, f) an IL-4 stimulating adjuvant and SDF-1α, g) a functional derivative, analogue or part of any of the substances a)-f) or h) a combination of any of the substances a)-g).

Use for the manufacture of a pharmaceutical for preventing or treating a Th1 cell-related disease by reducing the Th1/Th2 ratio, of a) IL-4 and SDF-1α, b) a stimulant of IL-4 and a stimulant of SDF-1α, c) an antagonist to IL-2 and an antagonist to SDF-1α, d) an inhibitor of Syk or NFAT1, e) a stimulant of ZAP-70 or NFAT2, f) an IL-4 stimulating adjuvant and SDF-1α, g) a functional derivative, analogue or part of any of the substances a)-f) or h) a combination of any of the substances a)-g).

A method of preventing or treating a Th1 cell-related disease by reducing the Th1/Th2 ratio comprising administering to a subject an effective dose of an active substance selected from the group consisting of a) IL-4 and SDF-1α, b) a stimulant of IL-4 and a stimulant of SDF-1α, c) an antagonist to IL-2 and an antagonist to SDF-1α, d) an inhibitor of Syk or NFAT1, e) a stimulant of ZAP-70 or NFAT2, f) an IL-4 stimulating adjuvant and SDF-1α, g) a functional derivative, analogue or part of any of the substances a)-f) or h) a combination of any of the substances a)-g).

A method of preventing or treating a Th1 cell-related disease by reducing the Th1/Th2 ratio comprising removing T helper cells from a subject, contacting ex vivo the cells with an effective dose of an active substance selected from the group consisting of a) IL-4 and SDF-1α, b) a stimulant of IL-4 and a stimulant of SDF-1α, c) an antagonist to IL-2 and an antagonist to SDF-1α, d) an inhibitor of Syk or NFAT1, e) a stimulant of ZAP-70 or NFAT2, f) an IL-4 stimulating adjuvant and SDF-1α, g) a functional derivative, analogue or part of any of the substances a)-f) or h) a combination of any of the substances a)-g).

A method as mentioned in one of the two previous paragraphs comprising subjecting the subject or recipient to be treated to a second treatment involving the manipulation of the immune system. The said second treatment involving the manipulation of the immune system amy be selected from the group consisting of a vaccination, antigen specific immunotherapy, allergen specific immunotherapy, nonspecific immunotherapy and an organ transplantation.

A pharmaceutical composition comprising an active substance selected from the group consisting of o) IL-2 and SDF-1α, p) a stimulant of IL-2 and a stimulant of SDF-1α, q) an antagonist to IL-4 and an antagonist to SDF-1α, r) an inhibitor of ZAP-70 or NFAT2, s) a stimulant of Syk or NFAT1, t) an IL-2 stimulating adjuvant and SDF-1α, u) a functional derivative, analogue or part of any of the substances o)-t) or v) a combination of any of the substances o)-u).

Use for the manufacture of a pharmaceutical for preventing or treating a Th2 cell-related disease by increasing the Th1/Th2 ratio, of o) IL-2 and SDF-1α, p) a stimulant of IL-2 and a stimulant of SDF-1α, q) an antagonist to IL-4 and an antagonist to SDF-1α, r) an inhibitor of ZAP-70 or NFAT2, s) a stimulant of Syk or NFAT1, t) an IL-2 stimulating adjuvant and SDF-1α, u) a functional derivative, analogue or part of any of the substances o)-t) or v) a combination of any of the substances o)-u).

A method of preventing or treating a Th2 cell-related disease by increasing the Th1/Th2 ratio comprising administering to a subject an effective dose of an active substance selected from the group consisting of o) IL-2 and SDF-1α, p) a stimulant of IL-2 and a stimulant of SDF-1α, q) an antagonist to IL-4 and an antagonist to SDF-1α, r) an inhibitor of ZAP-70 or NFAT2, s) a stimulant of Syk or NFAT1, t) an IL-2 stimulating adjuvant and SDF-1α, u) a functional derivative, analogue or part of any of the substances o)-t) or v) a combination of any of the substances o)-u).

A method of preventing or treating a Th2 cell-related disease by increasing is the Th1/Th2 ratio comprising removing T helper cells from a subject, contacting ex vivo the cells with an effective dose of an active substance selected from the group consisting of o) IL-2 and SDF-1α, p) a stimulant of IL-2 and a stimulant of SDF-1α, q) an antagonist to IL-4 and an antagonist to SDF-1α, r) an inhibitor of ZAP-70 or NFAT2, s) a stimulant of Syk or NFAT1, t) an IL-2 stimulating adjuvant and SDF-1α, u) a functional derivative, analogue or part of any of the substances o)-t) or v) a combination of any of the substances o)-u).

A method as mentioned in one of the two previous paragraphs comprising subjecting the subject or recipient to be treated to a second treatment involving the manipulation of the immune system. The said second treatment involving the manipulation of the immune system may be selected from the group consisting of a vaccination, antigen specific immunotherapy, allergen specific immunotherapy, nonspecific immunotherapy and organ transplantation.

An antisense peptide nucleic acid (PNA) that is complementary to a DNA molecule encoding the tyrosine kinase Syk or a part thereof. The Syk PNA preferably comprises 5-25, more preferably 10-20, most preferably 13-18 bases. Preferably, the Syk PNA has the sequence of SEQ ID NO. 01.

An antisense peptide nucleic acid (PNA) that is complementary to a DNA molecule encoding the tyrosine kinase Syk or a part thereof for preventing or treating a Th1 cell-related disease by reducing the Th1/Th2 ratio.

Use of an antisense peptide nucleic acid (PNA) that is complementary to a DNA molecule encoding the tyrosine kinase Syk or a part thereof for the manufacture of a medicament for preventing or treating a Th1 cell-related disease by reducing the Th1/Th2 ratio.

A method of preventing or treating a Th1 cell-related disease by reducing the Th1/Th2 ratio comprising administering to a subject an effective dose of an antisense peptide nucleic acid (PNA) that is complementary to a DNA molecule encoding the tyrosine kinase Syk or a part thereof.

An antisense peptide nucleic acid (PNA) that is complementary to a DNA molecule encoding the tyrosine kinase ZAP-70 or a part thereof. The ZAP-70 PNA preferably comprises 5-25, more preferably 10-20, most preferably 13-18 bases. Preferably, the ZAP-70 PNA has the sequence of SEQ ID NO. 02.

An antisense peptide nucleic acid (PNA) that is complementary to a DNA molecule encoding the tyrosine kinase ZAP-70 or a part thereof for preventing or treating a Th2 cell-related disease by increasing the Th1/Th2 ratio.

Use of an antisense peptide nucleic acid (PNA) that is complementary to a DNA molecule encoding the tyrosine kinase ZAP-70 or a part thereof for the manufacture of a medicament for preventing or treating a Th2 cell-related disease by increasing the Th1/Th2 ratio.

A method of preventing or treating a Th2 cell-related disease by increasing the Th1/Th2 ratio comprising administering to a subject an effective dose of an antisense peptide nucleic acid (PNA) that is complementary to a DNA molecule encoding the tyrosine kinase ZAP-70 or a part thereof.

An in vitro diagnostic method of evaluating the T helper cell profile of a subject comprising obtaining a T helper cell containing sample from the subject, measuring the level of phosphorylated Syk, phosphorylated ZAP-70, intranucleic NFAT1 and/or intranucleic NFAT2 in the sample and using the measuring results obtained to assess the Th1/Th2 level.

An in vitro method of testing the effect of a product or a method on the Th1/Th2 ratio, comprising obtaining a T helper cell containing culture with a known Th1/Th2 ratio, subjecting the T helper cells to the product or method, measuring the level of phosphorylated Syk, phosphorylated ZAP-70, intranucleic NFAT1 and/or intranucleic NFAT2 in the sample and using the measuring results obtained to assess the change in Th1/Th2 level.

A diagnostic test kit comprising one or more probes specific for binding to phosphorylated Syk, phosphorylated ZAP-70, intranucleic NFAT1 and/or intranucleic NFAT2, and optionally a detection system.

A method of producing a culture enriched in Th1 cells comprising obtaining a T helper cell containing sample, subjecting the sample to an active substance selected from the group consisting of a) IL-2 and SDF-1α, b) a stimulant of IL-2 and a stimulant of SDF-1α, c) an antagonist to IL-4 and an antagonist to SDF-1α, d) an inhibitor of ZAP-70 or NFAT2, e) a stimulant of Syk or NFAT1, f) a functional derivative, analogue or part of any of the substances a)-e) or g) a combination of any of the substances a)-f) to increase the Th1/Th2 ratio.

A method of producing a culture enriched in Th2 cells comprising obtaining a T helper cell containing sample, subjecting the sample to an active substance selected from the group consisting of a) IL-4 and SDF-1α, b) a stimulant of IL-4 and a stimulant of SDF-1α, c) an antagonist to IL-2 and an antagonist to SDF-1α, d) an inhibitor of Syk or NFAT1, e) a stimulant of ZAP-70 or NFAT2, f) a functional derivative, analogue or part of any of the substances a)-e) or g) a combination of any of the substances a)-f) to decrease the Th1/Th2 ratio.

Use of the culture produced by the method of claim 60 or 61 in in vitro or in vivo research and experiments.

### BRIEF DESCRIPTION OF THE FIGURES

FIG. 1A shows the activation of Syk in CB T cells either freshly isolated or stimulated with different combinations among IL-2 (10 ng/ml), IL-4 (10 ng/ml), and SDF-1α (100 ng/ml) as indicated using an immune complex kinase assay (KA) and immunoblotting (IB).

FIG. 1B shows the activation of ZAP-70 in CB T cells either freshly isolated or stimulated with different combinations among IL-2 (10 ng/ml), IL-4 (10 ng/ml), and SDF-1α (100 ng/ml) as indicated using an immune complex kinase assay (KA) and immunoblotting (IB).

FIG. 1C shows the activation of Cbl in CB T cells either freshly isolated or stimulated with different combinations among IL-2 (10 ng/ml), IL-4 (10 ng/ml), and SDF-1α (100 ng/ml) as indicated using an immune complex kinase assay (KA) and immunoblotting (IB).

FIG. 1D shows the activation of Cbl-b in CB T cells either freshly isolated or stimulated with different combinations among IL-2 (10 ng/ml), IL-4 (10 ng/ml), and SDF-1α (100 ng/ml) as indicated using an immune complex kinase assay (KA) and immunoblotting (IB).

FIG. 2A shows the blocking effect of PNA Syk antisense on the activation of Syk kinase using an immune complex kinase assay (KA) and immunoblotting (IB).

FIG. 2B shows the blocking effect of PNA ZAP-70 antisense on the activation of ZAP-70 kinase using an immune complex kinase assay (KA) and immunoblotting (IB).

FIG. 3A-D show the activation and identification of NFAT in CB T cells upon stimulation with different combinations among IL-2 (10 ng/ml), IL-4 (10 ng/ml), and SDF-1α (100 ng/ml).

FIG. 4 shows a schematic representation of regulatory pathways leading to Th1 and Th2 differentiation.

### DETAILED DESCRIPTION OF THE INVENTION

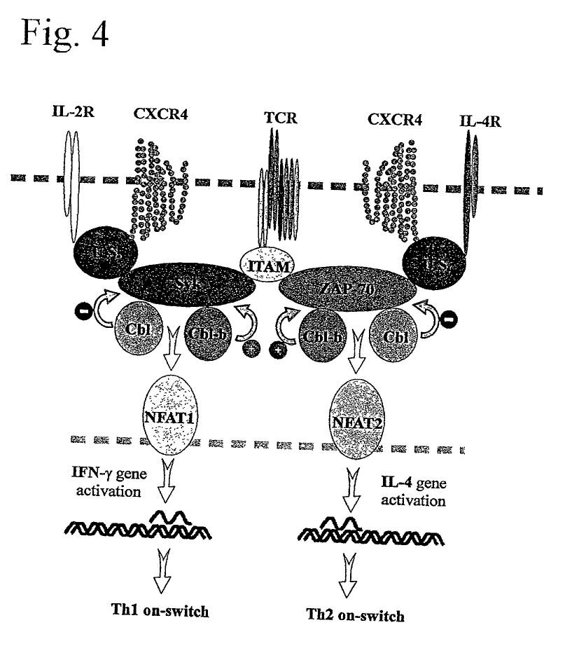

In the present invention, it was found that IL-2 or IL-4 in combination with SDF-1α switch cord blood (CB) CD4⁺ T cells to Th1 and Th2 type cells in a non-antigen-specific manner, respectively. This finding was demonstrated at both protein level and mRNA level. Parallelly, IL-2 or IL-4 in combination with SDF-1α induce a strong and persistent activation of Syk and ZAP-70 kinase within 8 days in CB CD4⁺ T cells, respectively. An attenuated pattern or an increased pattern of activity of Cbl kinase has been seen within 8 days in the cells stimulated with IL-2 and SDF-1α or with IL-4 and SDF-1α. In contrast, a strong and persistent activation of Cbl-b kinase has been seen within 8 days in the CB CD4⁺ T cells stimulated with either IL-4 and SDF-1α or IL-2 and SDF-1α. Syk or ZAP-70 PNA antisense completely abolish kinase activity and protein expression of Syk or ZAP-70 within 8 days in CB CD4⁺ T cells stimulated with IL-2 or IL-4 in combination with SDF-1α, respectively. Likewise, Syk or ZAP-70 PNA antisense selectively inhibit IFN-γ or IL-4 mRNA expression in CB CD4⁺ T cells induced by IL-2 or IL-4 combination with SDF-1α, implying that Syk and ZAP-70 kinase activation is essential for the Th1 or Th2 cell on-switch by IL-2 or IL-4 in combination with SDF-1α. Moreover, a selective and persistent NFAT1 or NFAT2 activation has been detected in (IL-2+SDF-1α)- or (IL-4+SDF-1α)-stimulated-CB T cells, indicating NFAT1 or NFAT2 also play an important and selective role in Th1 or Th2 cell on-switch. In conclusion, this invention reports an alternative signaling pathway in which IL-2 or IL-4 together with SDF-1α induce a selective and persistent phosphorylation of Syk or ZAP-70 kinase, resulting in on-switch of non-antigen-specific CD4⁺ T cells to Th1 or Th2 cells. A schematic view of the findings of this invention is given in FIG. 4.

As mentioned above, the experimental findings of the present invention is based on the use of cord blood CD4⁺ T cells, which were considered to be naive cells. It is believed that the findings for naive T cells are also valid for on-going immune responses, i.e. that it is possible to initiate an additional immune response, which is superimposed on the on-going response.

Diseases Treated

Th1-related Diseases

The Th1-related diseases comprise the following groups of diseases: infectious disesases, autoimmune diseases, delayed type hypersensitivity diseases and cancer.

The group of infectious diseases includes diseases caused by parasites and viruses, such as HIV.

The group of autoimmune diseases include encephalomyelopathic, demyelinating and other autoimune diseases.

Examples of encephalomyelopathic diseases include, but are not limited to, multiple sclerosis (MS); disseminated sclerosis; focal sclerosis; insular sclerosis; tabes dorsalis (posterior sclerosis); acute and chronic experimental allergic (or autoimmune) encephalomyelitis (EAE), which is an animal model of MS; Guillain-Barré syndrome; experimental allergic neuritis (an animal model of Guillain-Barré syndrome); acute disseminated encephalomyelitis; myalgic encephalomyelitis (benign and epidemic); viral encephalomyelitis; granulomatous encephalomyelitis; etc. Also included are animal diseases, such as but not limited to canine distemper; feline distemper; equine encephalomyelitis (eastern, Venezuelan, and western); avian encephalomyelitis; porcine encephalomyelitis; bovine encephalomyelitis; mouse encephalomyelitis; etc.

Examples of demyelinating diseases include, but are not limited to, multiple sclerosis (MS), disseminated sclerosis (DS), acute disseminated encephalomyelitis, progressive multifocal leukoencephalopati (PML), and subacute sclerotic panencephalitis (SSPE).

Examples of other auto-immune diseases include mutiple sclerosis (MS), polyartheritis nodosaasthma, hypersensitivity pneumonitis, interstitial lung disease, sarcoidosis, idiopathic pulmonary fibrosis, interstitial lung disease associated with Crohn's Disease or ulcerative colitis or Whipple's disease, interstitial lung disease associated with Wegeners granulomatosis or hypersensitivity vasculitis,

vasculitis syndromes, Hennoch-Schönleins purpura, Goodpastures syndrome, Wegeners granulomatosis,

renal diseases such as antibody mediated glomerulopathia as in acute glomerulonephritis, nephritis associated with systemic lupus erythematosus (SLE), nephritis associated with other systemic diseases such as Wegeners granulomatosis and Goodpastures syndrome and mixed connective tissue disease, chronic interstitial nephritis, chronic glomerulonephritis,

gastrointestinal diseases such as Crohn's Disease, Ulcerative colitis, coeliac disease, Whipple's disease, collagenous colitis, eosinophillic colitis, lymphatic colitis,

hepatobilliary diseases such as auto-immune hepatitis, alcohol induced hepatitis, periportal fibrosis, primary billiary cirrhosis, sclerosing colangitis,

skin diseases such as psoriasis, atopic dermatitis, eczema, allergic skin disease, progressive systemic sclerosis (scleroderma), exfoliating dermatitis, pemphigus vulgaris,

joint diseases such as rheumatoid arthritis (RA), ankylosing spondylitis, arthritis associated with psoriasis or inflammatory bowel disease,

muscoloskelletal diseases such as myastenia gravis (MG), polymyositis,

endocrine diseases such as insulin dependent diabetes mellitus (IDDM), auto-immune thyroiditis (Hashimoto), thyreotoxicosis, hyperthyroidism (Graves disease),

diseases of the hematopoetic system such as auto-immune anaemia, auto-immune thrombocytopenia,

cardiovascular diseases such as cardiomyopathia, vasculitis, cardiovascular disease associated with systemic diseases as systemic lupus erythematosus, polyarthritis nodosa, rheumatoid arthritis, scleroderma, sarcoidosis.

A noticeable characteristic of autoimmune diseases is their familial clustering and association with the expression of particular genes, in particular genes of class I and class II major histocompatibility complex (MHC). For example, a large proportion of MS patients have the HLA-DR2 haplotype (Beall S S, Concannon P, Charmley P, et al., J. Neuroimmunol.,v 21, p 59-66, 1989). Since not all subjects with a susceptible genotype develop the autoimmune disease, it appears that environmental factors also play a major role. For example, MS appears to be more common in subjects who live in temperate climate regions (Kurtzke J F, IN: Multiple Sclerosis, Hallpike J F, Adams C W M, Tourtelotte W W, editors, Williams and Wilkins, Baltimore, Md., 1983, p 49-95). It has long been speculated that the environmental factor of autoimmune diseases may be an infectious agent such as a virus. The etilogy of several human and animal diseases can be attributed to viral infection. For example, Theiler's murine encephalomyelitis is a demyelinating disease with clinical and pathological signs similar to EAE. Although antibodies are involved in some effector responses of autoimmune diseases, the triggering event in most cases begins with the activation of CD4 T-cells that are required for B cell maturation and clonal expansion.

The group of delayed type hypersensitivity include contact hypersensitivity to low molecular weight substances.

Th2-related Diseases

The Th2-related diseases comprise the following groups of diseases: Allergic diseases and cancer.

It is well known that genetically predisposed individuals become sensitised (allergic) to antigens originating from a variety of environmental sources, of which the individuals are exposed. The allergic reaction occurs when a previously sensitised individual is re-exposed to the same or a homologous allergen. Allergic responses range from hay fever, rhinoconductivitis, rhinitis and asthma to systemic anaphylaxis and death in response to e.g. bee or hornet sting or insect bite. The reaction is immediate and can be caused by a variety of atopic allergens such as compounds originating from grasses, trees, weeds, insects, (house dust) mites, food, drugs, chemicals and perfumes.

The group of allergic diseases includes hay fever, rhinoconjunctivitis, rhinitis and asthma.

The most common allergens, to which allergic reactions occur, include inhalation allergens originating i.a. from trees, grasses, herbs, fungi, house dust mites, storage mites, cockroaches and animal hair, feathers, and dandruff. Important pollen allergens from trees, grasses and herbs are such originating from the taxonomic orders of Fagales, Oleales and Pinales including i.a. birch (Betula), alder (Alnus), hazel (Corylus), hornbeam (Carpinus) and olive (Olea), the order of Poales including i.a. grasses of the genera Lolium, Phleum, Poa, Cynodon, Dactylis and Secale, the orders of Asterales and Urticales including i.a. herbs of the genera Ambrosia and Artemisia. Important inhalation allergens from fungi are i.a. such originating from the genera Alternaria and Cladosporium. Other important inhalation allergens are those from house dust mites of the genus Dermatophagoides, storage mites from the genus Lepidoglyphys destructor, those from cockroaches and those from mammals such as cat, dog, horse, cow, and bird. Also, allergic reactions towards stinging or biting insects such as those from the taxonomic order of Hymenoptera including bees, wasps, and ants are commonly observed. Specific allergen components are known to the person skilled in the art and include e.g. Bet v 1 (B. verrucosa, birch), Aln g 1 (Alnus glutinosa, alder), Cor a 1 (Corylus avelana, hazel) and Car b 1 (Carpinus betulus, hornbeam) of the Fagales order. Others are Cry j 1 (Pinales), Amb a 1 and 2, Art v 1 (Asterales), Par j 1 (Urticales), Ole e 1 (Oleales), Ave e 1, Cyn d 1, Dac g 1, Fes p 1, Hol l 1, Lol p 1 and 5, Pas n 1, Phi p 1 and 5, Poa p 1, 2 and 5, Sec c 1 and 5, and Sor h 1 (various grass pollens), Alt a 1 and Cha h 1 (fungi), Der f 1 and 2, Der p 1 and 2 (house dust mites, D. farinae and D. pteronyssinus, respectively), Lep d 1, Bla g 1 and 2, Per a 1 (cockroaches, Blatella germanica and Periplaneta americana, respectively), Fel d 1 (cat), Can f 1 (dog), Equ c 1, 2 and 3 (horse), Apis m 1 and 2 (honeybee), Ves g 1, 2 and 5, Pol a 1, 2 and 5 (all wasps) and Sol i 1, 2, 3 and 4 (fire ant), to mention the most common.

Th1- and Th2-related Cancer

In general, cancer cells may raise an immune response to antigens specifically expressed by the cancer cell. Since both Th1 and Th2 immune responses may drive strong inflammatory responses leading to cytotoxic eradication of tissue, modulation of Th1 or Th2 immune responses may be used in the treatment of cancer.

Cancers, which can be treated with the composition according to the invention can be histogenetically classified as malignant epithelial tumours, including carcinomas and adenocarcinomas, and as malignant non-epithelial tumours, including liposarcomas, fibrosarcomas, chondrosarcomas, osteosarcomas, leiomyosarcomas, rhabdomyosarcomas, gliomas, neuroblastomas, medulloblastomas, malignant melanoma, malignant meningioma, various leukemias, various myeloproliferative disorders, various lymphomas (Hodgkin's lymphoma and non-Hodgkin lymphoma), haemangiosarcoma, Kaposi's sarcoma, lymphangiosarcoma, malignant teratoma, dysgerminoma, seminoma, and choricarcinoma. Carcinomas and adenocarcinomas are the most abundant (accounting for approximately 90% of deaths from cancer) and are therefore interesting target diseases to treat/prevent according to the invention. The most important carcinomas and adenocarcinomas are those of the airways (espially of bronchial origin), of the breast, of the colorectum and of the stomach. However, also carcinomas and adenocarcinomas of the prostate, the ovary, of the lymphoid tissue and bone marrow, of the uterus, of the pancreas, of the esophagus, the urinary bladder, and the kidney cause a significant number of deaths and are therefore of interest.

The group of cancer diseases further includes Sezary's syndrome, cutaneous T-cell lymphoma, hepatic carcinoma and lung cancer.

Co-administration of Active Substance with Pathogenic Substance

In a preferred embodiment of the invention the pharmaceutical composition of the invention in addition to the active substance contains a pathogenic agent eliciting the disease to be treated. Such a pharmaceutical composition has the advantage that the deviation of the immune response is limited to the antigen specificity defined by the pathogenic substance.

Accordingly, the pharmaceutical composition of the invention may further contain a pathogenic substance eliciting the Th1-related disease to be treated, in particular an infectious agent eliciting an infectious disease or an antigen. The said antigen may be an autoantigen eliciting an autoimmune disease, a hapten or an allergen eliciting a delayed type hypersensitivity. Likewise, the pharmaceutical composition of the invention may further contain a pathogenic substance eliciting the Th2-related disease to be treated, in particular a parasite organism or part therof or an antigen. The antigen may be an allergen eliciting an allergic disease.

Active Substances

The active substances according to the present invention, i.e. substances a)-g) of claim 1 and o)-u) of claim 24 comprise both substances having an extracellular site of action, e.g. IL-4 and SDF-1α, and substances having an intracellular site of action, e.g. modulators of Syk, ZAP-70, NFAT1 and NFAT2.

The substances having an extracellular site of action typically bind to a receptor on the extracellular side of the cell membrane in a receptor-ligand system. In general, such a system may be inhibited by any substance binding to the receptor or ligand in a manner so that the binding between the receptor and ligand is impaired or prevented. Examples of such inhibitors are antibodies, low molecular organic substances and free receptors. A receptor-ligand system may be stimulated by any substance binding to the receptor and ligand in a manner so as to facilitate the binding between the receptor and ligand. Also, the receptor-ligand system may be stimulated by increasing the number of ligand molecules by addition thereof or the number of receptor molecules by adding a substance stimulating the intracellular expression of the receptor.

The substances having an intracellular site of action may be any substance capable of modulating the activity or expression of the substance.

In the following, the various active substances according to the present invention are described in more detail.

a) and o) IL-4/IL-2 and SDF-1α

IL-2, IL-4 and SDF-1α may be obtained by purification of the subsances from a biological material or by producing the substances using conventional recombinant techniques.

b) and p) Stimulants of IL-4/IL-2 and SDF-1α

Examples of stimulants of IL-2, IL-4 or SDF-1α are substances stimulating the intracellular activation and extracellular expression of IL-2, IL-4 or SDF-1α receptor. For a detailed account of substances capable of stimulating the expression of IL-2, IL-4 or SDF-1α receptor, reference is made to the below section with the heading “e) and s) Stimulants of Syk, ZAP-70, NFAT1 or NFAT2”, the content of which apply equally to this section.

c) and g) Antagonists of IL-2/IL-4 and SDF-1α

Antagonists to IL-2, IL-4 or SDF-1α include monoclonal and polyclonal antibodies to the IL-2, IL-4 or SDF-1α, both antibodies which bind to the cell receptor binding site of IL-2, IL-4 or SDF-1α and antibodies which bind to other parts of the IL-2, IL-4 or SDF-1α molecules in such a manner that the binding of the molecule to its receptor is reduced or hindered. Also, the antagonists to the ligands IL-2, IL-4 or SDF-1α include monoclonal and polyclonal antibodies to the receptors of IL-2, IL-4 or SDF-1α (CXCR4). The antibodies to the receptors may both be antibodies, which bind to the ligand binding site of the receptor and antibodies which bind to other parts of the receptor thereby reducing or preventing the binding of the ligand to the receptor.

The antagonists to IL-2, IL-4 or SDF-1α further includes peptides having a binding affinity for either the ligand IL-2, IL-4 or SDF-1α or their receptors, e.g. peptides comprising the binding site of one of the said molecules or a part thereof.

Furthermore, the antagonists of IL-2, IL-4 or SDF-1α may be low molecular compounds, e.g. organic compounds.

Also, the antagonists of IL-2, IL-4 or SDF-1α may be added as free receptors, i.e. receptors not bound to a T cell, and free ligands, which are able to bind to its receptor but unable to exert its effector function on T cells or only able to exert a reduced effector function.

Other examples of antagonists of IL-2, IL-4 or SDF-1α are substances inhibiting the intracellular expression of IL-2, IL-4 or SDF-1α receptor. For a detailed account of substances capable of inhibiting the expression of IL-2, IL-4 or SDF-1α receptor, reference is made to the below section with the heading “d) and r) Inhibitors of Syk, ZAP-70, NFAT1 or NFAT2”, the content of which apply equally to this section.

d) and r) Inhibitors of Syk, ZAP-70, NFAT1 and NFAT2

In the following, reference is made to WO 00/24245, which is included herein by this reference.

Inhibitory compounds of the invention can be, for example, intracellular binding molecules that act to specifically inhibit the expression or activity of Syk, ZAP-70, NFAT1 or NFAT2. As used herein, the term “intracellular binding molecule” is intended to include molecules that act intracellularly to inhibit the expression or activity of a protein by binding to the protein or to a nucleic acid (e.g., an mRNA molecule) that encodes the protein. Examples of intracellular binding molecules, described in further detail below, include antisense nucleic acids, intracellular antibodies, peptidic compounds that inhibit the interaction of Syk, ZAP-70, NFAT1 or NFAT2 with a target molecule (e.g., calcineurin) and chemical agents that specifically inhibit Syk, ZAP-70, NFAT1 or NFAT2 activity.

Further examples of inhibitory compounds include phosphatases and other enzymes and compounds capable of dephosphorylation.

i. Antisense Nucleic Acid Molecules

In one embodiment, an inhibitory compound of the invention is an antisense nucleic acid molecule that is complementary to a gene encoding Syk, ZAP-70, NFAT1 or NFAT2, or to a portion of said gene, or a recombinant expression vector encoding said antisense nucleic acid molecule. The antisense nucleic acid molecule may be a DNA, a RNA, a PNA, a LNA or a phosphorothioate. The use of antisense nucleic acids to downregulate the expression of a particular protein in a cell is well known in the art. An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. Preferably, an antisense nucleic acid is designed so as to be complementary to a region preceding or spanning the initiation codon on the coding strand or in the 3′ untranslated region of an mRNA.

Given the known nucleotide sequences for the coding strands of the Syk, ZAP-70, NFAT1 and NFAT2 genes (and thus the known sequences of the Syk, ZAP-70, NFAT1 and NFAT2 mRNAs), antisense nucleic acids of the invention can be designed according to the rules of Watson and Crick base pairing. The antisense nucleic acid molecule can be complementary to the entire coding region of a Syk, ZAP-70, NFAT1 or NFAT2 mRNA, but more preferably is an oligonucleotide which is antisense to only a portion of the coding or noncoding region of a Syk, ZAP-70, NFAT1 or NFAT2 mRNA. For example, the antisense oligonucleotide can be complementary to the region surrounding the translation start site of a Syk, ZAP-70, NFAT1 or NFAT2 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g. an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides an be used. Examples of modified nucleotides which can be used to generate the antisense nucleic acid include 5-fluorouracil, 5 bromouracil, 5 chlorouracil, 5-iodouracil, hypoxanthine, xanthine, 4-acetylcytosine, 5 (carboxyhydroxylmethyl) uracil, 5-carboxymethylamino-methyl-2-thiouridine, 5 carboxymethylaminomethyl-uracil, dihydrouracil, beta-D-galactosylqueosine, inosine, N6-isopentenyladenine, I-methylguanine, I-methylinosine, 2,2 dimethylguanine, 2 methyladenine, 2-methylguanine,3-methylcytosine, 5-methylcytosine, N6 adenine, 7 methylguanine, 5-methylaminomethyluracil, 5-methoxyaminomethyl-2 thiouracil, beta D-mannosylqueosine, 5′-methoxycarboxymethyluracil, 5-methoxyuracil, 2 methylthio N6-isopentenyladenine, uracil-5-oxyacetic acid (v), wybutoxosine, pseudouracil, queosine, 2-thiocytosine, 5-methyl-2-thiouracil, 2-thiouracil, 4 thiouracil, 5 methyluracil, uracil-5- oxyacetic acid methylester, uracil-5-oxyacetic acid (v), 5-methyl 2-thiouracil, 3-(3-amino-3-N-2-carboxypropyl) uracil, (acp3)w, and 2,6 diaminopurine. To inhibit Syk, ZAP-70, NFAT1 or NFAT2 expression in cells in culture, one or more antisense oligonucleotides can be added to cells in culture media.

Alternatively, an antisense nucleic acid can be produced biologically using an expression vector into which all or a portion of Syk, ZAP-70, NFAT1 or NFAT2 cDNA has been subcloned in an antisense orientation (i. e., nucleic acid transcribed from the inserted nucleic acid will be of an antisense orientation to a target nucleic acid of interest). Regulatory sequences operatively linked to a nucleic acid cloned in the antisense orientation can be chosen which direct the expression of the antisense RNA molecule in a cell of interest, for instance promoters and/or enhancers or other regulatory sequences can be chosen which direct constitutive, tissue specific or inducible expression of antisense RNA. The antisense expression vector is prepared according to standard recombinant DNA methods for constructing recombinant expression vectors, except that the Syk, ZAP-70, NFAT1 or NFAT2 cDNA (or portion thereof) is cloned into the vector in the antisense orientation. The antisense expression vector can be in the form of, for example, a recombinant plasmid, phagemid or attenuated virus. The antisense expression vector is introduced into cells using a standard transfection technique.

The antisense nucleic acid molecules of the invention are typically administered to a subject or generated in situ such that they hybridize with or bind to cellular mRNA and/or genomic DNA encoding a Syk, ZAP-70, NFAT1 or NFAT2 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of an antisense nucleic acid molecule of the invention includes direct injection at a tissue site. Alternatively, an antisense nucleic acid molecule can be modified to target selected cells and then administered systemically. For example, for systemic administration, an antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong pol 11 or pol III promoter are preferred.

In yet another embodiment, the antisense nucleic acid molecule of the invention is an α-anomenic nucleic acid molecule. An α-anomeric nucleic acid molecule forms specific double-stranded hybrids with complementary RNA in which, contrary to the usual P-units, the strands run parallel to each other.

The antisense nucleic acid molecule can also comprise a 2′-o methylribonucleotide or a chimeric RNA-DNA analogue.

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes) can be used to catalytically cleave Syk, ZAP-70, NFAT1 or NFAT2 mRNA transcripts to thereby inhibit translation of Syk, ZAP-70, NFAT1 or NFAT2 mRNAs. A ribozyme having specificity for a Syk-, ZAP-70-, NFAT1- or NFAT2-encoding nucleic acid can be designed based upon the nucleotide sequence of the Syk, ZAP-70, NFAT1 or NFAT2 cDNA. For example, a derivative of a Tetrahymena L-19 IVS RNA can be constructed in which the nucleotide sequence of the active site is complementary to the nucleotide sequence to be cleaved in a Syk-, ZAP-70-, NFAT1- or NFAT2-encoding mRNA.

Alternatively, Syk, ZAP-70, NFAT1 and NFAT2 gene expression can be inhibited by targeting nucleotide sequences complementary to a regulatory region of a Syk, ZAP-70, NFAT1 or NFAT2 gene (e.g., a Syk, ZAP-70, NFAT1 or NFAT2 promoter and/or enhancer) to form triple helical structures that prevent transcription of an NFAT1 gene in target cells.

Finally, Syk, ZAP-70, NFAT1 and NFAT2 can be inhibited by a nucleic acid encoding a catalytically inactive mutant of Syk, ZAP-70, NFAT1 and NFAT2.

ii. Intracellular Antibodies

Another type of inhibitory compound that can be used to inhibit the expression and/or activity of Syk, ZAP-70, NFAT1 or NFAT2 protein in a cell is an intracellular antibody specific for Syk, ZAP-70, NFAT1 or NFAT2 discussed herein. The use of intracellular antibodies to inhibit protein function in a cell is known in the art.

To inhibit protein activity using an intracellular antibody, a recombinant expression vector is prepared which encodes the antibody chains in a form such that, upon introduction of the vector into a cell, the antibody chains are expressed as a functional antibody in an intracellular compartment of the cell. For inhibition of transcription factor activity according to the inhibitory methods of the invention, preferably an intracellular antibody that specifically binds the transcription factor is expressed within the nucleus of the cell. Nuclear expression of an intracellular antibody can be accomplished by removing from the antibody light and heavy chain genes those nucleotide sequences that encode the N terminal hydrophobic leader sequences and adding nucleotide sequences encoding a nuclear localization signal at either the N- or C-terminus of the light and heavy chain genes. A preferred nuclear localization signal to be used for nuclear targeting of the intracellular antibody chains is the nuclear localization signal of SV40 Large T antigen.

To prepare an intracellular antibody expression vector, antibody light and heavy chain cDNAs encoding antibody chains specific for the target protein of interest, e.g. Syk, ZAP-70, NFAT1 or NFAT2 protein, is isolated, typically from a hybridoma that secretes a monoclonal antibody specific for Syk, ZAP-70, NFAT1 or NFAT2 protein. Preparation of antisera against Syk, ZAP-70, NFAT1 or NFAT2 protein has been described in the art. Anti-Syk, -ZAP-70, -NFAT1 or -NFAT2 antibodies can be prepared by immunizing a suitable subject, (e.g., rabbit, goat, mouse or other mammal) with a Syk, ZAP-70, NFAT1 or NFAT2 immunogen, respectively. An appropriate immunogenic preparation can contain, for examples, recombinantly expressed Syk, ZAP-70, NFAT1 or NFAT2 protein or a chemically synthesized Syk, ZAP-70, NFAT1 or NFAT2 peptide. The preparation can further include an adjuvant, such as Freund's complete or incomplete adjuvant, or similar immunostimulatory compound. Antibody-producing cells can be obtained from the subject and used to prepare monoclonal antibodies by standard techniques, such as the hybridoma technique. The technology for producing monoclonal antibody hybridomas is well known. Briefly, an immortal cell line (typically a myeloma) is fused to lymphocytes (typically splenocytes) from a mammal immunized with a Syk, ZAP-70, NFAT1 or NFAT2 protein immunogen as described above, and the culture supernatants of the resulting hybridoma cells are screened to identify a hybridoma producing a monoclonal antibody that binds specifically to the Syk, ZAP-70, NFAT1 or NFAT2 protein. Any of the many well known protocols used for fusing lymphocytes and immortalized cell lines can be applied for the purpose of generating an anti-Syk, -ZAP-70, -NFAT1 or -NFAT2 protein monoclonal antibody. Moreover, the ordinary skilled artisan will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines may be used as a fusion partner according to standard techniques, e.g., the P3-NSl/1-Ag4-1, P3-x63-Ag8.653 or Sp2/0-Agl4 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody that specifically binds the maf protein are identified by screening the hybridoma culture supernatants for such antibodies, e.g., using a standard ELISA assay.

Alternative to preparing monoclonal antibody-secreting hybridomas, a monoclonal antibody that binds to Syk, ZAP-70, NFAT1 or NFAT2 can be identified and isolated by screening a recombinant combinatorial immunoglobulin library (e.g., an antibody phage display library) with the protein, or a peptide thereof, to thereby isolate immunoglobulin library members that bind specifically to the protein. Kits for generating and screening phage display libraries are commercially available (e.g., the Pharmacia Recombinant Phage Antibody System, Catalog No. 27-9400-0 1; and the Stratagene SurfZ4pTM Phage Display Kit, Catalog No. 240612). Additionally, examples of methods and compounds particularly amenable for use in generating and screening antibody display library can be found in the literature.

Once a monoclonal antibody of interest specific for Syk, ZAP-70, NFAT1 or NFAT2 has been identified (e.g., either a hybridoma-derived monoclonal antibody or a recombinant antibody from a combinatorial library, including monoclonal antibodies to Syk, ZAP-70, NFAT1 or NFAT2 that are already known in the art), DNAs encoding the light and heavy chains of the monoclonal antibody are isolated by standard molecular biology techniques. For hybridoma derived antibodies, light and heavy chain cDNAs can be obtained, for example, by PCR amplification or cDNA library screening. For recombinant antibodies, such as from a phage display library, cDNA encoding the light and heavy chains can be recovered from the display package (e.g., phage) isolated during the library screening process. Nucleotide sequences of antibody light and heavy chain genes from which PCR primers or cDNA library probes can be prepared are known in the art. For example, many such sequences are disclosed in Kabat, E. A., et al. (1991) Sequences of Proteins of immunological Interest, Fifth Edition, U.S. Department of Health and Human Services, NIH Publication No. 91-3242 and in the “Vbase” human germline sequence database.

Once obtained, the antibody light and heavy chain sequences are cloned into a recombinant expression vector using standard methods. As discussed above, the sequences encoding the hydrophobic leaders of the light and heavy chains are removed and sequences encoding a nuclear localization signal (e.g., from SV40 Large T antigen) are linked in-frame to sequences encoding either the amino- or carboxy terminus of both the light and heavy chains. The expression vector can encode an intracellular antibody in one of several different forms. For example, in one embodiment, the vector encodes full-length antibody light and heavy chains such that a full-length antibody is expressed intracellularly. In another embodiment, the vector encodes a full-length light chain but only the VH/CHI region of the heavy chain such that a Fab fragment is expressed intracellularly. In the most preferred embodiment, the vector encodes a single chain antibody (scFv) wherein the variable regions of the light and heavy chains are linked by a flexible peptide linker (e.g., (GIY4Ser)3) and expressed as a single chain molecule. To inhibit transcription factor activity in a cell, the expression vector encoding the Syk, ZAP-70, NFAT1 or NFAT2-specific intracellular antibody is introduced into the cell by standard transfection methods as described hereinbefore.

iii. Syk-, ZAP-70-, NFAT1- or NFAT2-Derived Peptidic Compounds

In another embodiment, an inhibitory compound of the invention is a peptidic compound derived from the Syk, ZAP-70, NFAT1 or NFAT2 amino acid sequence. In particular, the inhibitory compound(s) comprises a portion of Syk, ZAP-70, NFAT1 or NFAT2 (or a mimetic thereof) that mediates interaction of Syk, ZAP-70, NFAT1 or NFAT2, respectively, with a target molecule such that contact of Syk, ZAP-70, NFAT1 or NFAT2 with this peptidic compound competitively inhibits the interaction of Syk, ZAP-70, NFAT1 or NFAT2, respectively, with the target molecule. In a preferred embodiment, the peptide compound is designed based on the region of NFAT1 that mediates interaction of NFAT1 with calcineurin. As described in Avramburu et al., (1998) Mol. Cell. 1:627-637 (expressly incorporated herein by reference), a conserved region in the amino terminus of NFAT proteins mediates interaction of the NFAT proteins with calcineurin and peptides spanning the region inhibit the ability of calcineurin to bind to and phosphorylate NFAT proteins, without affecting the phosphatase activity of calcineurin against other substrates. Moreover, when expressed intracellularly, peptide spanning this region inhibits NFAT dephosphorylation, nuclear translocation and NFAT-mediated gene expression in response to stimulation, thereby inhibiting NFAT dependent functions. The region of NFAT1 mediating interaction with calcineurin contains the conserved amino acid motif Ser-Pro-Arg-Ile-Glu-Ile-Thr (motif 1).

In a preferred embodiment, a NFAT inhibitory compound is a peptidic compound, which is prepared based on a calcineurin-interacting region of NFAT1. A peptide can be derived from the calcineurin-interacting region of NFAT1 having an amino acid sequence that comprises the above-mentioned amino acid motif 1. Alternatively, longer regions of human NFAT1 can be used such as a peptide, which spans the above-mentioned amino acid motif 1.

The peptidic compounds of the invention can be made intracellularly in cells (e.g., lymphoid cells) by introducing into the cells an expression vector encoding the peptide(s). Such expression vectors can be made by standard techniques, using, for example, oligonucleotides that encode one of the above discussed amino acid motifs. The peptide(s) can be expressed in intracellularly as a fusion with another protein or peptide (e.g., a GST fusion). Alternative to recombinant synthesis of the peptides in the cells, the peptides can be made by chemical synthesis using standard peptide synthesis techniques. Synthesized peptides can then be introduced into cells by a variety of means known in the art for introducing peptides into cells (e.g., liposome and the like). Recombinant methods of making NFAT inhibitory peptides, and methods using them to inhibit NFAT activity in cells, are described further in Avramburu et al., (1998) Mol. Cell. 1:627-637.

It also has been demonstrated that the region of NFAT1 that interacts with calcineurin is necessary for nuclear import of NFAT1 and for effective recognition and dephosphorylation such that mutation of this region inhibits NFAT1 activity (see Avramburu et al., (1998) Mol. Cell. 1:627-637). Thus, in another embodiment, NFAT1 activity can be inhibited by mutating the calcineurin-binding region in the amino terminus, comprising the above mentioned motif 1. The wildtype NFAT1 amino acid can be modified to the mutated sequence to create a mutated form of NFAT1 with reduced activity.

iv. Chemical Compounds

Other inhibitory agents that can be used to specifically inhibit the activity of Syk, ZAP-70, NFAT1 or NFAT2 proteins are chemical compounds that directly inhibit Syk, ZAP-70, NFAT1 or NFAT2 activity or inhibit the interaction between Syk, ZAP-70, NFAT1 or NFAT2 and target molecules. Such compounds can be identified using screening assays that select for such compounds.

Examples of ZAP-70 inhibitory chemical compounds are a 1,2,4-oxadiazole analog derived from L-glutamine, L-alanine, L-homo-Phenylalanine or L-serine (Vu, 2000); a mimetic of the bidentate ζ-ITAM peptide (Vu, 2000); a monodentate compound (Vu, 2000); a peptoid (Vu, 2000); a isothiazolone compound (Trevillyan et al., 1999); nocodazole (Huby et al., 1998); methyl-3-(N-isothiazolone)-2-thiophenecarboxylate (Trevillyan et al., 1999).

e) and s) Stimulants of Syk, ZAP-70, NFAT1 or NFAT2

Examples of stimulatory compounds include active Syk, ZAP-70, NFAT1 or NFAT2 protein, expression vectors encoding Syk, ZAP-70, NFAT1 or NFAT2 and chemical agents that specifically stimulate Syk, ZAP-70, NFAT1 or NFAT2 activity.

A preferred stimulatory compound is at least one nucleic acid molecule encoding Syk, ZAP-70, NFAT1 or NFAT2, wherein the nucleic acid molecule(s) is introduced into the subject in a form suitable for expression of the Syk, ZAP-70, NFAT1 or NFAT2 proteins in the cells of the subject. For example, Syk, ZAP-70, NFAT1 or NFAT2 cDNAs (full length or partial Syk, ZAP-70, NFAT1 or NFAT2 cDNA sequence) is cloned into a recombinant expression vector and the vector is transfected into cells using standard molecular biology techniques. The Syk, ZAP-70, NFAT1 or NFAT2 cDNAs can be obtained, for example, by amplification using the polymerase chain reaction (PCR) or by screening an appropriate cDNA library. The nucleotide sequences of Syk, ZAP-70, NFAT1 or NFAT2 cDNAs are known in the art and can be used for the design of PCR primers that allow for amplification of the cDNAs by standard PCR methods or for the design of a hybridization probe that can be used to screen a cDNA library using standard hybridization methods. Following isolation or amplification of Syk, ZAP-70, NFAT1 or NFAT2 cDNAs, the DNA fragments are introduced into one or more suitable expression vector, as described above. A single expression vector that carries both Syk and NFAT1 or ZAP-70 and NFAT2 coding sequences can be used, or two separate vectors can be used. Nucleic acid molecules encoding Syk, ZAP-70, NFAT1 or NFAT2 in the form suitable for expression of the Syk, ZAP-70, NFAT1 or NFAT2 in a host cell, can be prepared as described above using nucleotide sequences known in the art. The nucleotide sequences can be used for the design of PCR primers that allow for amplification of a cDNA by standard PCR methods or for the design of a hybridization probe that can be used to screen a cDNA library using standard hybridization methods.

Another form of a stimulatory compound for stimulating expression of Syk, ZAP-70, NFAT1 or NFAT2 in a cell is a chemical compound that specifically stimulates the expression or activity of endogenous Syk, ZAP-70, NFAT1 or NFAT2 in the cell. Bisperoxovanadium (bpV) is an example of a stimulant of Zap-70.

Stimulating chemical compounds can be identified using screening assays that select for compounds that stimulate the expression or activity of Syk, ZAP-70, NFAT1 or NFAT2.

f) and t) IL-4 and IL-2 Stimulating Adjuvants

The IL-4 stimulating adjuvant according to the present invention may be selected from Group A consisting of

Aluminium phosphate

Aluminium hydroxide

Alhydrogel

Calcium phosphate

Cholera Holotoxin

Cholera Toxin B Subunit

RehydragelHPA/LV

Polyphosphazene

Preferably, the Group A adjuvant is Alhydrogel or Calcium phosphate. The said Group A adjuvants are also referred to as Th2 cell stimulating adjuvants.

The IL-2 stimulating adjuvant according to the present invention may be selected from Group B consisting of

Avridine

Block copolymer P1205 and possibly other block copolymers

Threonyl-MDP

Specol (Marcol 52, Span 85, Tween 85)

QS-21

CpG molecules

Nonionic surfactant vesicles

Murapalmitine

Murametide

MPL (monophosphoryl lipid A)

E coli labile enterotoxin

Gamma Inulin

Freund's Complete Adjuvant

Freund's incomplete Adjuvant

Preferably, the Group B adjuvant is CpG molecules or MPL (monophosphoryl lipid A). The said Group B adjuvants are also referred to as Th1 cell stimulating adjuvants.

In general, the IL-4 stimulating adjuvant according to the invention may be any adjuvant, which gives rise to a cytokine response which is more Th2 cell skewed than the cytokine response of any of the Group B adjuvants mentioned above.

Likewise, the IL-2 stimulating adjuvant according to the invention may be any adjuvant, which gives rise to a cytokine response which is more Th1 cell skewed than the cytokine response of any of the Group A adjuvants mentioned above.

g) and u) Derivatives, Analogues or Parts of Substances a)f) and o)-t)

The derivatives, analogues or parts of substances a)-f) and o)-t) may be any derivative, analogue or part known in the prior art. For example, when the active substance is a protein, a part of the protein having at least a partly conserved functionality may be used. In particular, when the active substance is an antibody, a part of the antibody molecule comprising at least a part of its specificity may be used.

Pharmaceutical Composition

As discussed above, the active substance used in the present invention may i.a. be an organic substance, a peptide, a protein and a nucleic acid. Depending on the type of active substance used, the active substance may be formulated in any pharmaceutical composition known in the prior art, including pharmaceutical compositions for injection and for oral, parenteral, pulmonary and nasal administration.

Oral compositions include tablets, capsules, pills, troches or lozenges, cachets or pellets.

The pharmaceutical composition may include any pharmaceutical acceptable excipient known in the prior art, including diluents, preservatives, solubilizers, emulsifiers, colorants, flavoring agents, disintegrants, binders, antifrictional agents, glidants, surfactants, adjuvants and/or carriers, as described e.g. in Remington's Pharmaceutical Sciences, 18th Ed. (1990, Mack Publishing Co., Easton, Pa. 18042).

Method of Treatment

Preferably, the pharmaceutical composition of the invention is administered by parenteral injection or mucosal administration. Local administration is preferred over systemic administration to avoid any undesirable side effects of SDF-1α. In this connection the expression “local administration” means topical administration or local injection. By “local injection” means injection, wherein the systemic dissemination of the active substance is less than 50%, preferably 40%, more preferably 30%, more preferably 20% and most preferably 10%.

The method of treatment of the invention can be practiced either in vitro or in vivo (the latter is discussed further in the following subsection). For practicing the method in vitro, cells can be obtained from a subject by standard methods and incubated (i.e., cultured) in vitro with the pharmaceutical composition of the invention.

For example, lymphoid cells can be isolated from a subject, treated in vitro using a modulatory agent of the invention and then readministered to the same subject, or another subject tissue compatible with the donor of the cells. Accordingly, in another embodiment, the modulatory method of the invention comprises culturing cells in vitro with an active substance (modulator) and further comprises administering the cells to a subject to thereby modulate Th1/Th2 cell ratio in a subject. For administration of cells to a subject, it may be preferable to first remove residual compounds in the culture from the cells before administering them to the subject. This can be done for example by gradient centrifugation of the cells or by washing of the cells. For further discussion of ex vivo genetic modification of cells followed by readministration to a subject, see also U.S. Pat. No. 5,399,346 by W. F. Anderson et al.

In other embodiments, an active substancend is administered to a subject in vivo. For stimulatory or inhibitory agents that comprise nucleic acids (e.g., recombinant expression vectors encoding an active substance), the compounds can be introduced into cells of a subject using methods known in the art for introducing nucleic acid (e.g., DNA) into cells in vivo. Examples of such methods include:

Direct Injection: Naked DNA can be introduced into cells in vivo by directly injecting the DNA into the cells (see e.g., Acsadi et al. (1991) Nature 332:815-818; Wolff et al. (1990) Science 247:1465-1468). For example, a delivery apparatus (e.g., a “gene gun”) for injecting DNA into cells in vivo can be used. Such an apparatus is commercially available (e.g., from BioRad).

Receptor-Mediated DNA Uptake: Naked DNA can also be introduced into cells in vivo by complexing the DNA to a cation, such as polylysine, which is coupled to a ligand for a cell-surface receptor. Binding of the DNA-ligand complex to the receptor facilitates uptake of the DNA by receptor-mediated endocytosis. A DNA-ligand complex linked to adenovirus capsids which naturally disrupt endosomes, thereby releasing material into the cytoplasm can be used to avoid degradation of the complex by intracellular lysosomes.

Retroviruses: Defective retroviruses are well characterized for use in gene transfer for gene therapy purposes (for a review see Miller, A. D. (1990) Blood 76:27 1). A recombinant retrovirus can be constructed having a nucleotide sequence of interest incorporated into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include Y Crip, yCre, y2 and yAm. Retroviruses have been used to introduce a variety of genes into many different cell types, including epithelial cells, endothelial cells, lymphocytes, myoblasts, hepatocytes, bone marrow cells, in vitro and/or in vivo. Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

Adenoviruses: The genome of an adenovirus can be manipulated such that it encodes and expresses a gene product of interest but is inactivated in terms of its ability to replicate in a normal lytic viral life cycle. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium, endothelial cells, hepatocytes and muscle cells. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors. Most replication-defective adenoviral vectors currently in use are deleted for all or parts of the viral EI and E3 genes but retain as much as 80% of the adenoviral genetic material.

Adeno-Associated Viruses: Adeno-associated virus (AAV) is a naturally occurring defective virus that requires another virus, such as an adenovirus or a herpes virus, as a helper virus for efficient replication and a productive life cycle. It is also one of the few viruses that may integrate its DNA into non-dividing cells, and exhibits a high frequency of stable integration. Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AA vectors.

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay.

Diagnostic Test Kit

The diagnostic test kit comprises one or more probes for each compound to be measured. As probe any known probe suitable of binding to the compound to be measured, i.e. phosphorylated Syk, phosphorylated ZAP-70, intranucleic NFAT1 or intranucleic NFAT2, may be used, e.g. antibodies, nucleic acids, fragments therof etc. The probe may be labeled, e.g. with a tag.

Alternatively, the kit may comprise a detection system. As detection system any known detection system may be used. Preferably, the detection system comprises a detecting reagent, which is capable of binding to the probe, and which comprises a label compound. An example of such a detection system is a labelled antibody to the probe.

Definitions

In connection with the present invention the term “a stimulant of X” means any agent stimulating the expression and/or the activity of X directly or indirectly regardless of the mechanism involved.

In connection with the present invention the term “an inhibitor of X” means any agent inhibiting the expression and/or the activity of X directly or indirectly regardless of the mechanism involved.

The term “Th1 cell” means any CD4⁺ T helper 1 cell capable of secreting IL-2 and IFNγ.

The term “Th2 cell” means any CD4⁺ T helper 2 cell capable of secreting IL-4 and IL-5.

The expression “Th1 cell-related disease” means any disease, in which Th1 cells support, cause or mediate the disease process or in which Th1 cells are involved in curing or alleviating the symptoms of the disease.

The expression “Th2 cell-related disease” means any disease, in which Th2 cells support, cause or mediate the disease process or in which Th2 cells are involved in curing or alleviating the symptoms of the disease.

The term “pathogenic substance” means any substance, which elicits a disease in the subject.

The term “antigen” means any substance, which elicits an immunological disease in the subject.

The expression “immunological disease” means any disease, which is supported by the immunological response system.

The expressions “intranucleic NFAT1” and “intranucleic NFAT2” mean the NFAT1 and NFAT2, respectively, present in the nucleus of the T lymphocyte.

The term “adjuvant” means any substance, which nonspecifically enhances the immune response to antigen.

The expression “antisense nucleic acid molecule” means any nucleic acid molecule in the form of a DNA, a RNA, a PNA, a LNA or a phosphorothioate or derivatives, analogs or fragments thereof capable of down-regulating the expression of a particular protein encoded by a nucleic acid complementary of the antisense nucleic acid.

The expression “reducing the Th1/Th2 ratio” means that the level of Th1 cells is reduced and/or that the level of Th2 cells is increased. Correspondingly, the expression “increasing the Th1/Th2 ratio” means that the level of Th1 cells is increased and/or that the level of Th2 cells is reduced.

Abbreviations

IL-2: Interleukin 2

IL-4: Interleukin 4

SDF-1α: Stromal cell-derived factor-1α

Syk: Syk tyrosine kinase

ZAP-70: ZAP-70 tyrosine kinase (ζ chain associated protein 70 kD)

Cb1: casitas B-lineage lymphoma

Cb1-b: casitas B-lineage lymphoma-bregion

NFAT: Nuclear factor of activated T cells.

TCR: T cell receptor

CXCR4: CXC receptor 4

DNA: Deoxyribonucleic acid

RNA: Ribonucleic acid.

PNA: Peptide nucleic acid

LNA: Locked nucleic acid

### EXAMPLES

### Example 1

Materials and Methods

Cells

CD4⁺ T cells from umbilical cord blood of uncomplicated births were purified as described elsewhere (Jinquan et al., 1997). Briefly, mononuclear cells were separated from heparinized cord blood using Ficoll-Hypaque (Nycomed, Oslo, Norway). CD4⁺ T cells were purified using positive selection of Dynabeads (Dynal A/S, Norway) according to the manufacturer's instructions. The purity of CD4⁺ T cells was ≧96% measured by flow cytometry. All serum IgM were under detectable level.

Intracellular Immunofluorence Staining of Cytokines

As described elsewhere (Charmers et al., 1998), the cells were washed twice in PBS and then fixed and permeabilized using IntraPrep® (Coulter-ImmunoTech, Miami, Fla., USA) according to manufacture's instructions. For intracellular IL-4 staining, the cells were then incubated with the primary mouse anti human IL-4 mAb (10 μg/ml, R&D Systems, Oxon, UK) for 15 min at room temperature. Cells were washed twice and stained with FITC-conjugated goat anti mouse antibodies for 15 min at room temperature. For staining of a second cytokine, the stained cells were incubated with mouse IgG (300 μg/ml) for 30 min at room temperature to block free binding sites of the goat anti mouse antibodies. For the second intracellular cytokine INF-γ staining, the cells were then incubated with the primary mouse anti human INF-γ mAb (10 μg/ml, R&D Systems,) for 15 min at room temperature. Cells were washed twice and stained with PE-conjugated goat anti mouse antibodies for 15 min at room temperature. Cells were washed and resuspended in PBS containing 0.5% formaldehyde for FACS analysis.

Immunoprecipitation

As described elsewhere (Tamura et al., 1995), the cells (5×10⁶) were solubilized in 1 ml of cold TNE buffer consisting of 50 mM Tris-HCL (pH 8.0), 150 mM NaCl, 1% (v/v) Nonidet P-40 containing 20 mM EDTA, 10 μg/ml aprotinin, 0.4 mM sodium vanadate, and 10 mM sodium pyrophosphate. The cell lysates were centrifuged at 10,000×g for 5 min and the supernatants were precleared with protein G-Sepharose. The lysates were then incubated with 5 μg of rabbit anti-Syk (c-20), rabbit anti-ZAP-70 (LR), goat anti-Cbl (C-15), or goat anti-Cbl-b (C-20) (all from Santa Cruz BioTech. Inc., Santa Cruz, Calif., USA), at 4° C. for 1 h and the immune complexes were precipitated with protein G-Sepharose. For blotting, the immune complexes were washes five times with TNE buffer.

Immune Complex Kinase Assay

As described elsewhere (Tamura et al, 1995), the immune complex precipitated with protein G-Sepharose was washed four times with TNE buffer and four times with kinase buffer (50 mM HEPES-NaOH, pH 7.4, and 10 mM MnCl₂). The immunoprecipitates were suspended in 20 μL of kinase buffer containing 10 μCi of [γ-³²P] ATP and incubated at 30° C. for 30 min. The reaction was stopped by the addition of 15 μl of 3× sample buffer (195 mM Tris-HCl, pH 6.8, 9% SDS, 15% 2-ME, and 30% glycerol). Then the mixture was boiled for 5 min and subjected to 8% SDS-PAGE under reducing conditions, followed by autoradiography.

Immunoblotting

As described elsewhere (Tamura et al, 1995), proteins in immunoprecipitates were resolved by SDS-PAGE under reducing conditions and then transferred to polyvinylidene difluoride microporous membrane (Schleicher & Schuell Life Science, Dassel, Germany). The membrane was blocked in 5% BSA-TBS (20 mM Tris-HCL, pH 7.5, and 150 mM NaCl), and then incubated with anti-Syk, anti-ZAP-70, anti-Cbl, or anti-Cbl-b. Immunoblots were incubated with [γ-¹²⁵I] protein A (NEN® Life Science Products, Inc., Boston, Mass., USA). After the incubation the membrane was washed with TBS and followed by autoradiography.

Peptide Nucleic Acid (PNA) Antisense Assay

As previously described with a modification (Norton et al., 1996), purified CB T cells were permeabilized with a buffered solution containing a relatively low concentration of detergent (20 mM Tris-HCL, pH 8.3, 1.5 mM MgCL2, 68 mM KCL, 0.05% Tween 20, 1 mM ethylenebis (oxyethlene-nitrilo)tetraacetic acid, 5.0% glycerol, and 0.1 mM 4-(2-aminoethyl)benzenesulfonyl fluoride). The cells were then cultured in RPMI 1640 with 10% FCS in the presence of antisense PNA (PE Applied Biosystemss, Foster City, USA) at 2 μM with or without stimuli in period of time indicated. PNA sequences used were as follow:

| | |
|---|---|
| antisense Syk | |
| ₍₉₃₃₎5′-ATTTTTTGACATGGGA-3′₍₉₁₈₎. | (SEQ ID NO.01) |
| antisense ZAP-70 | |
| ₍₇₃₈₎5′-GTTTGCGCTCGGCCTC-3′₍₇₂₃₎. | (SEQ ID NO.02) |

For further assays, the cells were extensively washed prior to the procedures.

Real Time Quantitative Reverse Transcription (RT)-PCR Assay

All real time quantitative RT-PCR experiments were performed as described elsewhere (Heid et al., 1996; Kruse et al., 1997). Briefly, total RNA from CB T cells (2×10⁶) was prepared by using Quick Prep® Total RNA Extraction Kit (Pharmacia Biotech, USA) and any potential contaminating chromosomal DNA was digested with DNAse I according to the manufacturer's instructions. For reverse transcription, the RNA was reverse transcribed by using oligo (dT)12-18 and Superscript II reverse transcriptase (Life Technologies, Grand Island, USA), according to the manufacturer's instructions. Reverse transcription was performed for 60 min at 37° C., and any potential contaminating protein was denatured by incubation for 10 min at 95° C. Quantitative PCR was performed in special optical tubes in a 96 well microtiter plate (PE Applied Biosystems, Foster City, USA) format on an ABI PRISM® 7700 Sequence Detector Systems (PE Applied Biosystems), according to the manufacturer's instructions. By using SYBR® Green PCR Core Reagents Kit (Perkin Elmer Applied Biosystems, P/N 4304886), fluorescence signals were generated during each PCR cycle via the 5′ to 3′ endonuclease activity of AmpliTaq Gold (Kruse et al., 1997) to provide real time quantitative PCR information. The following sequences of the specific primers for γ IFN and IL-4 (DNA Technology, Aarhus, Denmark) were used:

| | |
|---|---|
| γIFN sense: | 5′-TGTAAGCCCCCAGAAACAGAAAG-3′, |
| γIFN antisense: | 5′-TTGCCCATCAAGAAACAGCAG-3′; |
| IL-4 sense: | 5′-TCACTCTTCACTCTTTTCTTCCCC-3′, |
| IL-4 antisense: | 5′-TCTTCCCACTTTGCTGTTCCTC-3′. |

These oligonucleotide sequences were designed by using software Primer ExpressTM 1.0 (PE Applied Biosystems). They span exon junctions in order to prevent from amplification of genomic DNA. Taqman® universal PCR master mix (PE Applied Biosystems) containing Passive Reference 1 (ROX) was used to normalize for non-PCR-related fluctuations in fluorescence signal. The standard DNA template with known amounts of molecules (1.0×10³, 2.0×10³, 4.0×10³, 1.0×10⁴, 2.0×10⁴, 1.0×10⁵ per well) and “no template” controls were used to create standard curves. All unknown cDNAs were diluted to contain equal amounts of β-actin cDNA. The standards, “no template” controls and unknown samples were added in a total volume of 50 μl per reaction. PCR retain conditions were 2 min at 50° C., 10 min at 95° C., 40 cycles with 15 s at 95° C., 60 s at 60° C. for each amplification. Potential PCR product contamination was digested by uracil-N-glycosylase (UNG) since dTTP is substituted by dUTP (Kruse et al., 1997). All PCR experiments were performed with a hot start. In the reaction system, UNG and AmpliTaq Gold (PE Applied Biosystems) were applied according to the manufacturer's instructions (Heid et al., 1996; Kruse et al., 1997). In order to analyze data of PCR products two terms were used to express the results: ΔRn representing the normalized reporter signal minus the baseline signal established in the first few cycles PCR; CT (threshold cycle) representing the PCR cycle at which an increase in reporter fluorescence signal above a baseline can first be detected.

Electrophoretic Mobility Shift Assay (EMSA)

Nuclear extracts were prepared as described by McCaffrey (McCaffrey et al., 1992; Aramburu et al., 1995). Briefly, 400 μl ice-cold Dignam buffer A and then 25 μl 10% NP-40 were added into the cells (5×10⁶). The cells were vortexed and centrifuged (9000 rpm, 30 s, 4° C.). Pelleted nuclei were lysed in 50 μl Dignam buffer C, centrifuged (12,000 rpm, 10 min, 4° C.), and the resulting supernatants were diluted (1:1) with Dignam buffer D. Double-stranded synthetic oligonucleotide DNA probes were end-labeled with [γ-32P] ATP (5.000 Ci/mmol) and T4 polynucleotide kinase (Amersham Pharmacia Biotech Inc., UK). The sequences of the oligonucleotide probes used (5′ to 3′, one strand) were (Jain et al., 1993; McCaffrey et al., 1993): human IL-2 distal NFAT site (N FAT hulL-2) GGAGGAAAAACTGTTTCATACAGAAGG (binding sequences was underlined). EMSA reactions were performed at room temperature in a final volume of 25 μl. Nuclear extracts (3 μg protein per reaction volume) were incubated for 15 min in binding buffer (Aramburu et al., 1995), followed by addition of 0.5 ng 32P-labeled probes to react for 15 min, and the samples were electrophoresed on nondenatured 5% polyacrylamide gels in 0.25 TBE buffer. In some experiments, the mAbs or pAbs used for supershift (all at 10 μg/ml) were incubated with the nuclear extracts on ice for 30 min.

Results

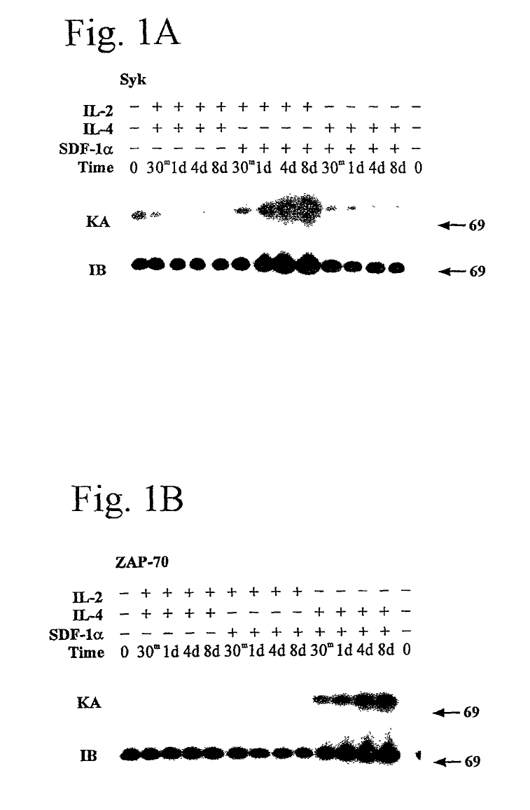

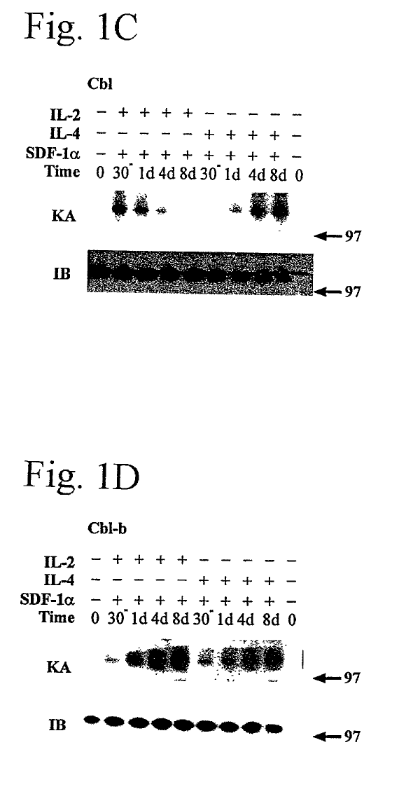

The results are shown in Table 1, 2 and 3 and in FIGS. 1A, 1B, 1C, 1D, 2A, 2B, 3A, 3B, 3C and 3D.

CB CD4⁺ T Cells are Switched into Th1 and Th2 T Cells

TABLE 1
Intracellular Th1 and Th2 cytokine detection by flow cytometry

| | Day 1 | | | Day 2 | | | Day 4 | | | Day 8 | | |
|---|---|---|---|---|---|---|---|---|---|---|---|---|
| | | | IFN-γ | | | IFN-γ | | | IFN-γ | | | IFN-γ |
| Stimuli^{a} | IFN-γ^{b} | IL-4 | +IL-4 | IFN-γ | IL-4 | +IL-4 | IFN-γ | IL-4 | +IL-4 | IFN-γ | IL-4 | +IL-4 |
| IL-2 + SDF-1α | 11.4^{b} | 13.6 | 18.8 | 35.4 | 0.9 | 8.4 | 60.9 | 5.0 | 3.5 | 84.0 | 0.2 | 0.4 |
| lL-4 + SDF-1α | 9.7 | 9.9 | 6.2 | 0.1 | 21.1 | 0.4 | 6.2 | 62.2 | 6.9 | 0.1 | 90.3 | 0.1 |
| IL-2 + SDF-1α + Ab^{c} | 14.2 | 6.7 | 14.9 | 18.0 | 6.6 | 2.8 | 4.7 | 13.1 | 14.9 | 0.4 | 1.6 | 9.5 |
| IL-4 + SDF-1α + Ab | 9.2 | 2.8 | 21.2 | 14.2 | 0.5 | 5.4 | 11.2 | 29.1 | 15.8 | 2.8 | 0.6 | 12.3 |
| IL-2 + SDF-1α + I^{d} | 16.7 | 9.8 | 11.2 | 33.9 | 0.5 | 5.5 | 59.3 | 16.8 | 3.7 | 85.3 | 0.1 | 0.2 |
| IL-4 + SDF-1α + I | 5.6 | 12.3 | 11.0 | 5.5 | 23.1 | 5.6 | 12.9 | 57.2 | 7.8 | 3.9 | 84.3 | 2.5 |
| Freshly isolated | 8.5 | 12.1 | 9.7 | N.D.^{e} | N.D. | N.D. | N.D. | N.D. | N.D. | N.D. | N.D. | N.D. |

^{a}The CB T cells were stimulated with different combinations among IL-2 (10 ng/ml), IL-4 (10 ng/ml), and SDF-1α (100 ng/ml) as indicated before intracellular cytokine assay as as described in Materials and Methods.
^{b}The numbers listed in the table are in percentage detected with intracellular cytokine assay as described in Materials and Methods.
Th1 and Th2 cytokines are IFN-γ, IL-4 or IFN-γ and IL-4.
^{c}CXCR4 mouse mAb at 5 μg/ml.
^{d}Isotype Ig matched with CXCR4 mouse mAb at 5 μg/ml.
^{e}N.D. no determination. The listed data are from a single representative experiment of five performed.

As will appear from the data shown in Table 1, the CD4⁺ T cells from normal CB seem to be “undifferentiated and unprimed” showing naive Th pattern. In freshly isolated CB CD4⁺ T cells IFN-γ and IL-4 double positive are 9.7%, whereas IFN-γ or IL-4 single positive are 8.5% or 12.1%, respectively. After 8 days of stimulation with IL-2 and SDF-1α, the cells have been switched to Th1 pattern in terms of expression of IFN-γ (84%), whereas the stimulation with IL-4 and SDF-1α leads the CB T cells to express Th2 pattern (90.3%). None of IL-2, IL-4 and SDF-1α alone nor combination of IL-2 and IL-4 has shown such function (data not shown). No significant difference has been seen in terms of cellular proliferation between CB CD4⁺ T cells stimulated with IL-2 or IL-4 together with SDF-1α and CB CD4⁺ T cells cultured without stimulus within 8 days as detected by [³H]thymidine incorporation into DNA assay (our unpublished results). The cells cultured without stimulation have not been seen any significant chang in terms of expression of intracellular cytokines during 8 days (data not shown). Interestingly, CXCR4 mAb can significantly block such on-switch, whereas isotype Ig can not do it. Moreover, CD3 mAb has been used to substitute SDF-1α to stimulate CB T cells in which we have not been able to induce such pattern on-switch (data not shown).

TABLE 2
The mRNA expression of IFN-γ and IL-4

| | IFN- | | IL-4 | |
|---|---|---|---|---|
| Stimuli | Ct | Copies | Ct | IL-4 |
| IL-2 + SDF-1α | 18.9 | 1.0 × 10⁴ | 24.7 | 3.6 × 10² |
| IL-4 + SDF-1α | 27.4 | 7.8 × 10¹ | 19.1 | 1.3 × 10⁴ |
| Freshly | 23.5 | 7.3 × 10² | 21.7 | 2.1 × 10³ |

Table 2: Real time detection and amplification of cDNA of IFN-γ and IL-4 in CB T cells. IL-2 and IL-4 were used at 10 ng/ml and SDF-1α was used at 100 ng/ml. The shown values are representatives of four similar experiments performed.

The mRNA expression of IFN-γ or IL-4 in CB T cells by real time quantitative RT-PCR has also been examined. As shown in Table 2, there are approximately 7.3×10² copies for IFN-γ in freshly isolated CB T cells, 1.0×10⁴ copies for IFN-γ in the cells stimulated for 8 days with IL-2 and SDF-1α, and 7.8×10¹ copies for IFN-γ in the cells stimulated for 8 days with IL-4 and SDF-1α, respectively. As also shown in Table 2, there are approximately 2.1×10³ copies for IL-4 in freshly isolated CB T cells, 3.6×10² copies for IL-4 in the cells stimulated for 8 days with IL-2 and SDF-1α, and 1.3×10⁴ copies for IL-4 in the cells stimulated for 8 days with IL4 and SDF-1′, respectively. A linear relationship between C_{T} and log starting quantity of standard DNA template or target cDNA (IFN-γ and IL-4) has been detected (data not shown). The correlation coefficients are ≧0.99.

Persistent Activation of Syk or ZAP-70 is Essential

FIG. 1 shows the activation of Syk (A), ZAP-70 (B), Cbl (C), and Cbl-b (D) kinase in the CB T cells. The cells were either freshly isolated or stimulated with different combinations among IL-2 (10 ng/ml), IL-4 (10 ng/ml), and SDF-1α (100 ng/ml) as indicated. KA represents immune complex kinase assay and IB represents immunoblotting. The cells were stimulated for different time intervals indicated, lysed, and immunoprecipitated with rabbit anti-Syk pAb, rabbit anti-ZAP-70 pAb, goat anti-Cbl pAb or goat anti-Cbl-b pAb as described in Materials and Methods. The immunoprecipitates were subjected to kinase reactions or immunoblotted with each antibody indicated as described in Materials and Methods. The leftmost lanes were where antibodies have been used alone.

As shown in FIG. 1, IL-2 and SDF-1α together has induced an activation of Syk kinase within 30 min. A strong and persistent phosphorylation of Syk kinase has been seen in 4d and 8d stimulation with IL-2 and SDF-1α, whereas neither combination of IL-2 and IL-4 nor combination of IL-4 and SDF-1α has induced Syk kinase activation compared with the level in freshly isolated CB T cells (FIG. 1A). In contrast, IL-4 and SDF-1α together has induced a phosphorylation of ZAP-70 kinase within 30 min. A strong and persistent activation of ZAP-70 has been seen in 4d and 8d stimulation with IL-4 and SDF-1α, whereas neither combination of IL-2 and IL-4 nor combination of IL-2 and SDF-1α has induced ZAP-70 activation compared with the level in freshly isolated CB T cells (FIG. 1B). No band has been observed in lanes where normal rabbit serum has been used in immunoprecipitation assays (data not shown).

Furthermore, the activation of adaptor proteins Cbl and Cbl-b in CB T cells has been investigated. As shown in FIGS. 1C and 1D, there are no detectable kinase activities of Cbl and Cbl-b kinase in freshly isolated CB T cells. IL-2 and SDF-1α together has induced a strong activation of Cbl within 30 min. An attenuating pattern of activity of Cbl kinase has been seen in 4d and 8d stimulation with IL-2 and SDF-1α, whereas an increasing pattern of activity of Cbl kinase has been seen in 4d and 8d stimulation with IL-4 and SDF-1α (FIG. 1C). In contrast, IL-2 and SDF-1α together has induced an weak activation of Cbl-b kinase within 30 min. A strong and persistent activation of Cbl-b kinase has been seen in 4d and 8d stimulation with either IL-2 and SDF-1α or IL-4 and SDF-1α (FIG. 1D). Since they were reported to have contrary effect on ZAP-70 kinase, the final outcome of the regulatory effect of Cbl and Cbl-b depends upon the balance between the activity of the two kinases. No band has been observed in lanes where goat serum has been used in immunoprecipitation assays (data not shown).

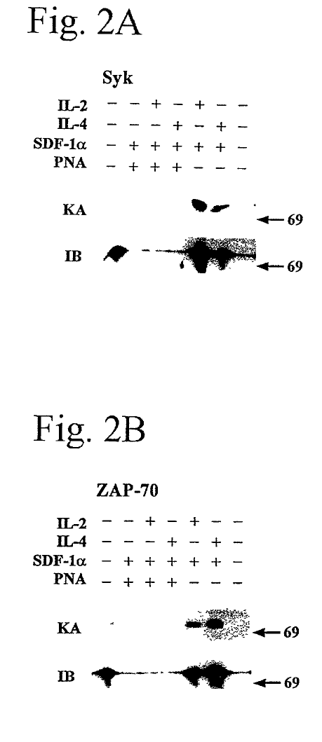

FIG. 2 shows the blocking effects of PNA Syk and PNA ZAP-70 antisenses on the activation of Syk kinase and ZAP-70 kinase. In A and B, the cells were purified CB T cells, which were permeabilized prior to further assays, and cultured in the presence or absence of PNA antisense indicated as described in Materials and Methods and with other stimuli indicated for 8 days. As described in connection with FIG. 1, the cells were then lysed, immunoprecipitated, and subjected to kinase reactions (KA) or immunoblotted (IB). The leftmost lanes were where antibodies have been used alone. In order to confirm the above observation, PNA antisense assays have been conducted. Syk PNA antisense and ZAP-70 PNA antisense also significantly inhibit kinase activity and protein expression of Syk and ZAP-70 in non-stimulated CB T cells in culture within 8 days, respectively (FIG. 2). Syk PNA antisense can significantly reduce Syk kinase activity within one day (data not shown), and completely abolish Syk kinase activity within 8 days in (IL-2+SDF-1α)- and (IL-4+SDF-1α)-stimulated CB T cells (FIG. 2A). The total amount of Syk protein is also severely reduced. Likewise, ZAP-70 PNA antisense can significantly reduce ZAP-70 kinase activity within one day (data not shown), and completely abolish ZAP-70 kinase activity within 8 days in (IL-2+SDF-1α)- and (IL-4+SDF-1α)-stimulated CB T cells (FIG. 2B). The ZAP-70 protein is also completely eliminated. No band has been observed in lanes where rabbit serum has been used in immunoprecipitation assays (data not shown).

TABLE 3
The mRNA expression of IFN-γ and IL-4:
effects of Syk PNA and Zap-70 PNA

| | IFN-γ | | IL-4 | |
|---|---|---|---|---|
| Stimuli | Ct | Copies | Ct | IL-4 |
| Syk PNA + | 23.1 | 4.5 × 10² | 25.6 | 3.6 × 10² |
| IL-2 + SDF-1α | | | | |
| Zap-70 PNA + | 16.6 | 1.1 × 10⁵ | 24.7 | 8.5 × 10² |
| IL-2 + SDF-1α | | | | |
| Syk PNA + | 22.4 | 1.2 × 10³ | 18.9 | 1.9 × 10⁴ |
| IL-4 + SDF-1α | | | | |
| Zap-70 PNA + | 25.1 | 1.4 × 10² | 26.1 | 6.1 × 10¹ |
| IL-4 + SDF-1α | | | | |

Table 3: Real time detection and amplification of cDNA of IFN-γ and IL-4 in CB T cells. IL-2 and IL-4 were used at 10 ng/ml and SDF-1α was used at 100 ng/ml. The shown values are representatives of four similar experiments performed.

In parallel, Syk PNA antisense has dramatically inhibited IFN-γ mRNA expression (4.5×10² copies) induced by IL-2 and SDF-1α (Table 3), whereas it has not significantly changed IFN-γ mRNA expression (1.2×10³ copies) induced by IL-4 and SDF-1α. In contrast, ZAP-70 PNA antisense has not such ability to inhibit IFN-γ mRNA expression (1.1×10⁵ copies) induced by IL-2 and SDF-1α, whereas it has not significantly changed IFN-γ mRNA expression (1.4×10² copies) induced by IL-4 and SDF-1α either. In parallel again, ZAP-70 PNA antisense has dramatically inhibited IL-4 mRNA expression (6.1×10¹ copies) induced by IL-4 and SDF-1α (Table 3), whereas it has not significantly changed IL-4 mRNA expression (1.8×10² copies) induced by IL-2 and SDF-1α. In contrast, Syk PNA antisense has no such ability to inhibit IL-4 mRNA expression (1.9×10⁴ copies) induced by IL-4 and SDF-1α, whereas it has not significantly changed IL-4 mRNA expression (8.5×10² copies) induced by IL-2 and SDF-1α either. Syk PNA antisense and ZAP-70 PNA antisense also significantly inhibit mRNA expression of IFN-γ and IL-4 in non-stimulated CB T cells in culture within 8 days, respectively (data not shown). The correlation coefficients in experiments for real time mRNA quantification are ≧0.97. Taken together, these results indicate that Syk kinase activation is essential for the Th1 cell on-switch induced by IL-2 and SDF-1α, and that ZAP-70 kinase activation is essential for the Th2 cell on-switch induced by IL-4 and SDF-1α.

Persistent Activation of NFAT1 or NFAT2 is also Important

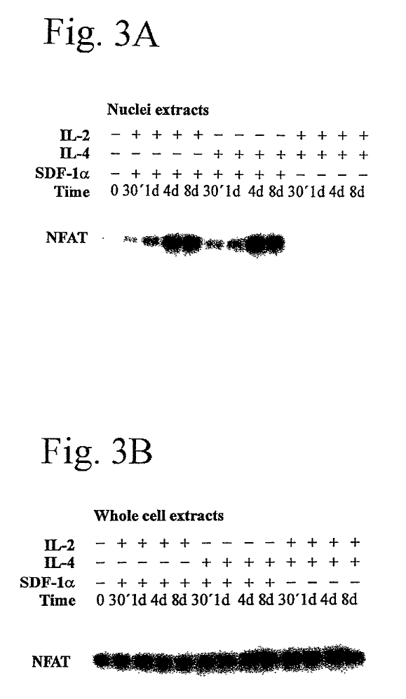

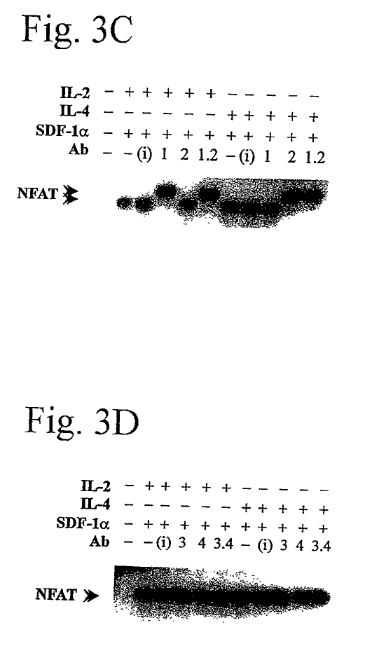

FIG. 3 shows activation and identification of NFAT in CB T cells upon stimulation with different combinations among IL-2 (10 ng/ml), IL-4 (10 ng/ml), and SDF-1α (100 ng/ml). The tested materials were either nuclei extracts (A, C and D) or whole cell extracts (B). The cells were freshly isolated or stimulated with different stimuli for each time intervals as indicated. NFAT activation was assessed by EMSA using a ³²P-labeled NFAT hulL-2 probe as described in Material and Methods. In A and B, the positions of NFAT were indicated. The identification of presence of NFAT1, NFAT2, NFAT3, and NFAT4 were analyzed by using Abs either towards NFAT1 or/and NFAT2 (C) or towards NFAT3 or/and NFAT4 (D) and EMSA in supershift assays as described in Material and Methods. Before addition of a ³²P-labeled NFAT hulL-2 probe, identical aliquots of samples were incubated without (−), or with isotype mouse IgG or goat serum (i), mouse NFAT1 mAb (1), mouse NFAT2 mAb (2), NFAT1 mAb plus NFAT2 mAb (1.2), or goat NFAT3 pAb (3), goat NFAT4 pAb (4), NFAT3 pAb plus NFAT4 pAb (3.4), NFAT binding to ³²P-labeled NFAT hulL-2 and the supershift induced in the presence of specific Ab are indicated with lower and upper arrowheads, respectively.

In order to further clarify downstream events after Syk or ZAP-70 activation, we have next investigated activation of NFAT in (IL-2+SDF-1α)- or (IL-4+SDF-1α)-stimulated CB T cells. In FIG. 3A, there is no detectable NFAT in nuclear extracts of freshly isolated CB T cells. After stimulation with IL-2+SDF-1α, IL-4+SDF-1α, or IL-2+IL-4 for 30 min, NFAT hulL-2 have been detected in nuclear extracts of CB T cells, which indicates that the stimulation with these combinations can induce NFAT activation ensuing a translocation of NFAT in CB T cells. However, IL-2+IL-4 only induce weak activation of NFAT in terms of translocation of NFAT from cytoplasm to nuclear. (IL-2+SDF-1α) or (IL-4+SDF-1α) stimulations have induced strong and persistent NFAT activations in CB T cells in 1d, 4d and 8d, whereas this phenomenon has not been seen in CB T cells stimulated with (IL-2+IL-4) (FIG. 3A). In contrast, there are almost equal NFAT protein seen in whole cell extracts of CB T cells either freshly isolated or after stimulations with different combinations (IL-2+SDF-1α, IL-4+SDF-1α, or IL-2+IL-4) (FIG. 3B), indicating that the stimulations with different combinations only induce activation of NFAT, but not new synthesis of NFAT. In FIG. 3C, NFAT complex has been detected with NFAT hulL-2 probe in nuclear extracts of CB T cells after stimulation with IL-2 and SDF-1α for 8d. This complex has been supershifted by the anti-NFAT1 mAb. Neither isotype mouse antibody nor anti-NFAT2 mAb can affect the relative mobility of the NFAT complex in nuclear extracts of (IL-2+SDF-1α)-stimulated CB T cells. In contrast, NFAT complex has been supershifted by the anti-NFAT2 mAb in nuclear extracts of CB T cells after stimulation with IL-4 and SDF-1α. Neither isotype mouse antibody nor anti-NFAT1 mAb can affect the relative mobility of the NFAT complex in nuclear extracts of (IL-4+SDF-1α)-stimulated CB T cells. These results confirm that only NFAT1, but not NFAT2, is present in nuclear extracts of (IL-2+SDF-1α)-stimulated CB T cells. These results also confirm that only NFAT2, but not NFAT1, is present in nuclear extracts of (IL-4+SDF-1α)-stimulated CB T cells. In FIG. 3D, NFAT complex, detected with in nuclear extracts of (IL-2+SDF-1α)- or (IL-4+SDF-1α)-stimulated CB T cells, has not been supershifted by either anti-NFAT3 pAb or anti-NFAT4 pAb at all. These results confirm that neither NFAT3 nor NFAT4 is present in nuclear extracts of (IL-2+SDF-1α)- or (IL-4+SDF-1α)-stimulated CB T cells.

Discussion

The balance between Th1 and Th2 cytokine profiles could modify the immune response at sites of inflammation (Coffman et al., 1999). For instance, allergic inflammation is a Th2-associated disease (Casolaro et al., 1996). Th2 cytokines play an essential role in the initiation, development, progression and termination of allergic inflammatory process. The determination of the expression and dynamics of Th1 and Th2 cytokine will be critical for the diagnosis and prognosis of Th1- and Th2-associated diseases. One important aspect should be considered that Th1 and Th2 clones were first isolated from hyperimmunized mice (O'Garra et al., 1998) or during chronic diseases in humans (Romagnani et al., 1994; Sher et al., 1992). So far human Th1 and Th2 T cells have been isolated from peripheral blood, draining lymph nodes and affected tissues during chronic infectious diseases and allergy (Romagnani et al., 1994). Although their ability to influence chronic disease or pathological process by their production of high levels of regulatory cytokines is not in doubt, to what numerical extent Th1 and Th2 cells dominate such in vivo responses is as yet not clear (O'Garra et al., 1998). Th1 and Th2 subsets develop from the same T cell precursor, which is a mature, naive CD4⁺ T cell producing mainly IL-2 upon antigen-specific stimulation (O'Garra et al., 1998). The factors determining Th1 and Th2 differentiation from this precursor are cytokines present at the initiation of the immune response at the stage of ligation of the TCR (Abbas et al., 1996). The present data showing that IL-2 or IL-4 in combination with SDF-1α can switch non-antigen-specific CB CD4⁺ T cells to Th1 or Th2 cells certainly raises the question whether SDF-1α has a mitogen-like function in terms of direction of Th1 and Th2 cell formation. If not, the question can be extended to whether the stage of antigen specifying in Th1 and Th2 cell formation is absolutely necessary. Parallel to induction of Th1 and Th2 formation, SDF-1α together with IL-2 or IL-4 induce a persistent and selective activation of Syk and ZAP-70 kinase, subsequently cause downstream activation of other elements of signal transduction including Cbl family and NFAT family. Thus, these data seem to point out an alternative pathway that some chemokines (SDF-1α) together with Th directive cytokines can selectively initiate an alternative TCR-associated tyrosine kinases phosphorylation and subsequently activate downstream elements including regulators, and such a series of activation of elements in signal transduction and the balances among them lead to an on-switch of Th1 or Th2 polarization. A schematic view for this system is illustrated in FIG. 4.

TCR-mediated signal transduction is critical for T cell development. Syk and ZAP-70, belonging to the Syk kinase family and associated with TCR, are different both in terms of expression and activity despite their clear structural resemblance (Chu et al., 1998). Although there is some evidence indicating that the kinases in the Syk family are selectively involved in activation of Th1 and Th2 cells (Tamura et al., 1995; Faith et al., 1999), the signaling pathways leading to polarization of Th1 and Th2 cells are not very clear until now. The current study provides the first direct evidence that persistent Syk kinase activation induced by IL-2 and SDF-1α leads to Th1 cell on-switch, whereas persistent ZAP-70 activation induced by IL-4 and SDF-1α trigger Th2 cell on-switch. Thus, it is evidence that the differences between Syk and ZAP-70 exist not only in terms of expression and activity, but also in terms of function. Chemokines regulate a number of biological processes, including trafficking of diverse leukocytes and proliferation of myeloid progenitor cells. Th1 and Th2 cells express different sets of chemokine receptors. Although there is suggestion of a cross-talk and reciprocal regulation between CXCR4 and the TCR (Peacock et al., 1999), the present study is the first to report that SDF-1α in combination with IL-2 or IL-4 selectively and specifically induce an accumulating and persistent Syk or ZAP-70 kinase activation, subsequently on-switch Th1 or Th2 cells.

In the early stage of their development, both Th1 and Th2 cells are interconvertible, but with repeated stimulation both populations become irreversible. The biochemical basis of this reversibility or stability may be the regulated transcription of cytokine genes or the expression of cytokine receptors (Abbas et al., 1996). Some researchers hold a view that progenitor Th cells differentiate into Th1 or Th2 cell clone upon antigen stimulation and cytokine environment in vivo. Some clinical symptoms seem not to sustain such opinion. For example, allergic asthma in which Th2 cytokines play a crucial and predominate role, could be onset within seconds, but during the remission, the balance between Th1 and Th2 cytokines could be upheld reasonably. Based on the present results it may be hypothesised that the resting Th cells could be entirely and quickly switched to Th1 or Th2 cells upon antigen stimulation and cytokine stimulation in vivo, which occurs locally in an early phase and systematically in a late phase. Newborn children have a low level of trigger-able Th1 and Th2 cells. These cells become memory Th1 and Th2 cells during development of naive Th cells. Memory Th1 and Th2 cells, which are in balance in normal condition in term of number and ability of synthesis and secretion of cytokines, may exist in vivo after exposure of antigen. Memory Th1 and Th2 cells, perhaps genetic “ill” Th1 and Th2 cells, together with other immune cells such as macrophages, provide an autocrine cytokine environment in situ, where Th1- or Th2-polarized diseases occur.

### LIST OF REFERENCES

1. Abbas, A. K., Murphy, K. M. and Sher, A. (1996) Functional diversity of helper T lymphocytes. Nature, 383, 787-793.

2. al-Ramadi, B. K., Nakamura, T., Leitenberg, D. and Bothwell, A. L. (1996) Deficient expression of p56(Ick) in Th2 cells leads to partial TCR signaling and a dysregulation in lymphokine mRNA levels. J. Immunol., 157, 4751-4761.

3. Aramburu, J., Azzoni, L., Rao, A. and Perussia, B. (1995) Activation and expression of the nuclear factor of activated T cells, NFATp and NFATc, in human natural killer cells: regulation upon CD16 ligand binding. J. Exp. Med., 182, 801-810.

4. Bird, J. J., Brown, D. R., Mullen, A. C., Moskowitz, N. H., Mahowald, M. A., Sider, J. R., Gajewski, T. F., Wang, C. R. and Reiner, S. L. (1998) Helper T cell differentiation is controlled by the cell cycle. Immunity, 9, 229-237.

5. Casolaro, V., Georas, S. N., Song, Z. and Ono, S. J. (1996) Biology and genetics of atopic disease. Curr. Opin. Immunol., 8, 796-803.

6. Charmers, I. M., Janossy, G., Contreras, M. and Navarrete, C. (1998) Intracellular cytokine profile of cord and adult blood lymphocytes. Blood, 92, 11-18.

7. Chu, D. H., Morita, C. T. and Weiss, A. (1998) The Syk family of protein tyrosine kinases in T-cell activation and development. Immunol, Rev., 165, 167-180.

8. Coffman, R. L. and Reiner, S. L. (1999) Instruction, selection, or tampering with the odds? Science, 284, 1283-1285.

9. Deckert, M., Tartare-Deckert, S., Hernandez, J., Rottapel, R. and Altman, A. (1998) Adaptor function for the Syk kinases-interacting protein 3BP2 in IL-2 gene activation. Immunity, 9, 595-605.

10. Faith, A., Akdis, C. A., Akdis, M., Simon, H. U., and Blaser, K. (1997) Defective TCR stimulation in anergized type 2 T helper cells correlates with abrogated p56(Ick) and ZAP-70 tyrosine kinase activities. J. Immunol., 159, 53-60.

11. Faith, A., Akdis, C. A., Akdis, M., Joss, A., Wymann, D. and Blaser, K. (1999) An altered peptide ligand specifically inhibits Th2 cytokine synthesis by abrogating TCR signaling. J. Immunol., 162, 1836-1842.

12. Heid, C. A., Stevens, J., Livak, K. J. and William, P.M. (1996) Real time quantitative PCR. Genome. Res., 6, 986-994.

13. Hodge, M. R.; Ranger A. M.; de la Brousse F. C.; Hoey T.; Grusby M. J.; Glimcher L. H. Hyperproliferation and Dysregulation of IL-4 Expression in NF-Atp-Deficient Mice. Immunity, Vol. 4, 397-405, April 1996.

14. Huby, R. D. J., Weiss, A. and Ley, S. C. 1998. Nocodazole Inhibits Signal Transduction by the T Cell antigen Receptor. The Journal of Biological Chemistry.

15. Jain, J.,. Miner, Z. and Rao, A. (1993) Analysis of the preexisting and nuclear forms of nuclear factor of activated T cells. J. Immunol., 151, 837-848.

16. Jinquan, T., Moller, B., Storgaard, M., Mukaida, N., Bonde, J., Grunnet, N., Black, F. T., Larsen, C. G., Matsushima, K. and Thestrup-Pedersen, K. (1997) Chemotaxis and IL-8 receptor expression in B cells from normal and HIV-infected subjects. J. Immunol., 158, 475-484.

17. Jourdan, P., Abbal, C., Nora, N., Hori, T., Uchiyama, T., Vendrell, J., Bousquet, J., Taylor, N., Péne, J. and Yssel H. 1998. Cutting Edge: IL-4 Induces Functional Cell-Surface Expression of CXCR4 on Human T cells. Cutting Edge.

18. Kruse, N., Pette, M., Toyka, K. and Rieckmann,P. (1997) Quantification of cytokine mRNA expression by RT PCR in samples of previously frozen blood. J. Immunol. Methods., 210,195-203.

19. McCaffrey, P. G., Jain, J., Jamieson, C., Sen, R. and Rao, A. (1992) A T cell nuclear factor resembling NF-AT binds to an NF-KB site and to the conserved lymphokine promoter sequence “cytokine-1.” J. Bio. Chem., 267,1864-1871.

20. McCaffrey, P. G., Luo, C., Kerppola, T. K, Jain, J., Badalian, T. M, Ho, A. M., Burgeon, E., Lane, W. S., Lambert, J. N., Curran, T. et al. (1993) Isolation of the cyclosporin-sensitive T cell transcription factor NFATp. Science, 262, 750-754.

21. Meng, W., Sawasdikosol, S., Burakoff, S. J. and Eck, M. J. (1999) Structure of the amino-terminal domain of Cbl complexed to its binding site on ZAP-70 kinase. Nature, 398, 84-90.

22. Norton, J. C., Piatyszek, M. A., Wright, W. E., Shay, J. W. and Corey, D. R., (1996) Inhibition of human telomerase activity by peptide nucleic acids. Nat. Biotechnol., 14, 615-619.

23. O'Garra, A. (1998) Cytokines induce the development of functionally heterogeneous T helper cell subsets. Immunity, 8, 275-283.

24. Ota, Y. and Samelson, L. E. (1997) The product of the proto-oncogene c-cbl: a negative regulator of the Syk tyrosine kinase. Science, 276, 418-420.

25. Peacock, J. W. and Jirik, F. R. (1999) TCR activation inhibits chemotaxis toward stromal cell-derived factor-1: evidence for reciprocal regulation between CXCR4 and the TCR. J. Immuno., 162, 215-223.

26. Sher, A. and Coffman, R. L. (1 992) Regulation of immunity to parasites by T cells and T cell-derived cytokines. Annu. Rev. Immunol., 10, 385-409.

27. Ranger, A. M., Hodge, M. R., Gravallese, E. M., Oukka, M., Davidson, L., Alt, F. W., de la Brousse, F. C., Hoey, T., Grusby, M. and Gilmcher, L. H. Delayed Lymphoid Repopulation with Defects in IL-4-Driven Responses Produced by Inactivation of NF-ATc. Immunity.

28. Romagnani, S. (1994) Lymphokine production by human T cells in disease states. Annu. Rev. Immunol., 12, 227-257.

29. Smith, J. A., Tang, Q. and Bluestone, J. A. (1998) Partial TCR signals delivered by FcR-nonbinding anti-CD3 monoclonal antibodies differentially regulate individual Th subsets. J. Immunol., 160, 4841-4849.

30. Tamura, T., Nakano H., Nagase, H., Morokata, T., Igarashi, O., Oshimi, Y., Miyazaki, S., Nariuchi, H. (1995) Early activation signal transduction pathways of Th1 and Th2 cell clones stimulated with anti-CD3. Roles of protein tyrosine kinases in the signal for IL-2 and IL-4 production. J. Immunol., 155,4692-4701.

31. Trevillyan, J. M., Chiou, X. G., Ballaron, S. J., Tang, Q. M., Buko, A., Sheets, M. P., Smith, M. L., Putman, C, B., Wiedeman, P., Tu, N., Madar, D., Smith, H. T., Gubbins, E. J., Warrior, U. P., Chen, Y., Mollison K. W., Faltynek, C. R. and Djuric, S. W. 1999. Inhibition of p56^{lck} Tyrosine Kinase by Isothiazolones. Archives of Biochemistry and Biophysics.

32. Viola, J. P. B., Kiani, A., Bozza, P. T. and Rao A. 1998. Regulation of Allergic Infallmation and Eosinophil Recruitment in Mice, Lacking the Transcription Factor NFAT1: Role of Interleukin-4 (IL-4) and IL-5. Rapid Communication.

33. Vu, C. B. 2000. Recent Advances in the Design and Synthesis of SH2 Inhibitors of Src, Grb2 and ZAP-70. Current Medicinal Chemitry.

34. Yoshida, H., Nishina, H., Takimoto, H., Marengére, L. E. M., Wakeham, A. C., Bouchard, D., Kong, Y., Ohteki, T., Shahinian, A., Bachmann, M., Ohashi, P. S., Penninger, J. M., Crabtree, G. R. and Mak, T. W. 1998. The Transcription Factor NF-Atc1 Regulates Lymphocyte Proliferation and Th2 Cytokine Production. Immunity.

35. Zhang, Z., Elly, C., Qiu, L., Altman, A. and Liu, Y. C. (1999) A direct interaction between the adaptor protein Cbl-b and the kinase zap-70 induces a positive signal in T cells. Curr. Biol., 9, 203-206.

1. A pharmaceutical composition for preventing or treating a Th1 cell-related disease in a human or an animal by reducing the Th1/Th2 ratio comprising an active substance selected from the group consisting of a) IL-4 and SDF-1α, b) a stimulant of IL-4 and a stimulant of SDF-1α, c) an antagonist to IL-2 and an antagonist to SDF-1α, d) an inhibitor of Syk or NFAT1, e) a stimulant of ZAP-70 or NFAT2, f) an IL-4 stimulating adjuvant and SDF-1α, g) a functional derivative, analogue or part of any of the substances a)-f) or h) a combination of any of the substances a)-g).

2. The composition according to claim 1, wherein the Th1 cell-related disease is an infectious disease.

3. The composition according to claim 1, wherein the Th1 cell-related disease is an autoimmune disease.

4. The composition according to claim 1, wherein the Th1 cell-related disease is a delayed type hypersensitivity.

5. The composition according to claim 1, wherein the Th1 cell-related disease is a cancer.

6. The composition according to claim 1 further comprising a pathogenic substance eliciting the Th1-related disease to be treated.

7. The composition according to claim 6, wherein the pathogenic substance is an infectious agent eliciting an infectious disease.

8. The composition according to claim 6, wherein the pathogenic substance is an antigen.

9. The composition according to claim 8, wherein the antigen is an autoantigen eliciting an autoimmune disease.

10. The composition according to claim 8, wherein the antigen is a hapten or an allergen eliciting a delayed type hypersensitivity.

11. The composition according to claim 1, wherein substance b) is a stimulant of the bonding between IL-4 or SDF-1α and their respective receptors; or a stimulant of the expression of IL-4 or SDF-1α receptor.

12. The composition according to claim 1, wherein substance c) is an antibody to IL-2 or SDF-1α or one of their receptors; a peptide having binding affinity for IL-2 or SDF-1α or one of their receptors; a low molecular compound, a free IL-2 or SDF-1α receptor; IL-2 or SDF-1α with reduced ability of exerting its effector function on the T cell; or an inhibitor of the expression of IL-2 or SDF-1α receptor.

13. The composition according to claim 1, wherein substance d) is i) an antisense nucleic acid molecule, ii) an intracellular antibody, iii) a Syk- or NFAT1-derived peptidic compound or iv) a chemical compound that specifically inhibits Syk or NFAT1.

14. The composition according to claim 13, wherein the antisense strand is a DNA, a RNA, a PNA, a LNA or a phosphorothioate.

15. The composition according to claim 1, wherein substance e) is active ZAP-70 or NFAT2 protein, expression vectors encoding ZAP-70 or NFAT2 and chemical agents that specifically stimulate ZAP-70 or NFAT2 activity.

16. The composition according to claim 1, wherein substance e) is bisperoxovanadium (bpV).

17. The composition according to claim 1, wherein substance f) is Calcium phosphate or Alhydrogel.

18. A pharmaceutical composition comprising an active substance selected from the group consisting of a) IL-4 and SDF-1α, b) a stimulant of IL-4 and a stimulant of SDF-1α, c) an antagonist to IL-2 and an antagonist to SDF-1α, d) an inhibitor of Syk or NFAT1, e) a stimulant of ZAP-70 or NFAT2, f) an IL-4 stimulating adjuvant and SDF-1α, g) a functional derivative, analogue or part of any of the substances a)-f) or h) a combination of any of the substances a)-g).

19. A method for the manufacture of a pharmaceutical for preventing or treating a Th1 cell-related disease which comprises reducing the Th1/Th2 ratio, of a) IL-4 and SDF-1α, b) a stimulant of IL-4 and a stimulant of SDF-1α, c) an antagonist to IL-2 and an antagonist to SDF-1α, d) an inhibitor of Syk or NFAT1, e) a stimulant of ZAP-70 or NFAT2, f) an IL-4 stimulating adjuvant and SDF-1α, g) a functional derivative, analogue or part of any of the substances a)-f) or h) a combination of any of the substances a)-g).

20. A method of preventing or treating a Th1 cell-related disease by reducing the Th1/Th2 ratio comprising administering to a subject an effective dose of an active substance selected from the group consisting of a) IL-4 and SDF-1α, b) a stimulant of IL-4 and a stimulant of SDF-1α, c) an antagonist to IL-2 and an antagonist to SDF-1α, d) an inhibitor of Syk or NFAT1, e) a stimulant of ZAP-70 or NFAT2, f) an IL-4 stimulating adjuvant and SDF-1α, g) a functional derivative, analogue or part of any of the substances a)-f) or h) a combination of any of the substances a)-g).

21. A method of preventing or treating a Th1 cell-related disease by reducing the Th1/Th2 ratio comprising removing T helper cells from a subject, contacting ex vivo the cells with an effective dose of an active substance selected from the group consisting of a) IL-4 and SDF-1α, b) a stimulant of IL-4 and a stimulant of SDF-1α, c) an antagonist to IL-2 and an antagonist to SDF-1α, d) an inhibitor of Syk or NFAT1, e) a stimulant of ZAP-70 or NFAT2, f) an IL-4 stimulating adjuvant and SDF-1α, g) a functional derivative, analogue or part of any of the substances a)-f) or h) a combination of any of the substances a)-g).

22. A method of claim 20 comprising subjecting the subject or recipient to be treated to a second treatment involving the manipulation of the immune system.

23. A method according to claim 22, wherein the second treatment involving the manipulation of the immune system is selected from the group consisting of a vaccination, antigen specific immunotherapy, allergen specific immunotherapy, nonspecific immunotherapy and an organ transplantation.

24. A pharmaceutical composition for preventing or treating a Th2 cell-related disease in a human or an animal by increasing the Th1/Th2 ratio comprising an active substance selected from the group consisting of o) IL-2 and SDF-1α, p) a stimulant of IL-2 and a stimulant of SDF-1α, q) an antagonist to IL-4 and an antagonist to SDF-1α, r) an inhibitor of ZAP-70 or NFAT2, s) a stimulant of Syk or NFAT1, t) an IL-2 stimulating adjuvant and SDF-1α, u) a functional derivative, analogue or part of any of the substances o)-t) or v) a combination of any of the substances o)-u).

25. The composition according to claim 24, wherein the Th2 cell-related disease is an allergic disease.

26. The composition according to claim 25, wherein the allergic disease is selected from the group consisting of hay fever, rhinoconjunctivitis, rhinitis and asthma.

27. The composition according to claim 24, wherein the Th2 cell-related disease is a cancer.

28. The composition according to claim 24 further comprising a pathogenic substance eliciting the Th2-related disease to be treated.

29. The composition according to claim 28, wherein the pathogenic substance is a parasite organism or part thereof.

30. The composition according to claim 28, wherein the pathogenic substance is an antigen.

31. The composition according to claim 30, wherein the antigen is an allergen eliciting an allergic disease.

32. The composition according to claim 24, wherein substance p) is a stimulant of the bond between IL-2 or SDF-1α and their respective receptors; or
a stimulant of the expression of IL-2 or SDF-1α receptor.

33. The composition according to claim 24, wherein substance q) is an antibody to IL-4 or SDF-1α or one of their receptors; a peptide having binding affinity for IL-4 or SDF-1α or one of their receptors; a low molecular compound, a free IL-4 or SDF-1α receptor; IL-4 or SDF-1α with reduced ability of exerting its effector function on the T cell; or an inhibitor of the expression of IL-4 or SDF-1α receptor.

34. The composition according to claim 24, wherein substance r) is i) an antisense nucleic acid molecule, ii) an intracellular antibody or iii) a ZAP-70- or NFAT2-derived peptidic compound or iv) a chemical compound that specifically inhibits ZAP-70 or NFAT2.

35. The composition according to claim 34, wherein substance r) is a 1,2,4-oxadiazole analog derived from L-glutamine, L-alanine, L-homo-Phenylalanine or L-serine; a mimetic of the bidentate ζ-ITAM peptide; a monodentate compound; a peptoid; a isothiazolone compound; nocodazole; methyl-3-(N-isothiazolone)-2-thiophenecarboxylate; DNA encoding a catalytically inactive mutant of ZAP-70.

36. The composition according to claim 34, wherein the antisense nucleic acid molecule is a DNA, a RNA, a PNA, a LNA or a phosphorothioate.

37. The composition according to claim 24, wherein substance s) is active Syk or NFAT1 protein, expression vectors encoding Syk or NFAT1 and chemical agents that specifically stimulate Syk or NFAT1 activity.

38. The composition according to claim 24, wherein substance t) is CpG molecules or MPL (monophosphoryl lipid A).

39. A pharmaceutical composition comprising an active substance selected from the group consisting of o) IL-2 and SDF-1α, p) a stimulant of IL-2 and a stimulant of SDF-1α, q) an antagonist to IL-4 and an antagonist to SDF-1α, r) an inhibitor of ZAP-70 or NFAT2, s) a stimulant of Syk or NFAT1, t) an IL-2 stimulating adjuvant and SDF-1α, u) a functional derivative, analogue or part of any of the substances o)-t) or v) a combination of any of the substances o)-u).

40. A method for the manufacture of a pharmaceutical for preventing or treating a Th2 cell-related disease which comprises increasing the Th1/Th2 ratio, of o) IL-2 and SDF-1α, p) a stimulant of IL-2 and a stimulant of SDF-1α, q) an antagonist to IL-4 and an antagonist to SDF-1α, r) an inhibitor of ZAP -70 or NFAT2, s) a stimulant of Syk or NFAT1, t) an IL-2 stimulating adjuvant and SDF-1α, u) a functional derivative, analogue or part of any of the substances o)-t) or v) a combination of any of the substances o)-u).

41. A method of preventing or treating a Th2 cell-related disease by increasing the Th1/Th2 ratio comprising administering to a subject an effective dose of an active substance selected from the group consisting of o) IL-2 and SDF-1α, p) a stimulant of IL-2 and a stimulant of SDF-1α, q) an antagonist to IL-4 and an antagonist to SDF-1α, r) an inhibitor of ZAP-70 or NFAT2, s) a stimulant of Syk or NFAT1, t) an IL-2 stimulating adjuvant and SDF-1α, u) a functional derivative, analogue or part of any of the substances o)-t) or v) a combination of any of the substances o)-u).

42. A method of preventing or treating a Th2 cell-related disease by increasing the Th1/Th2 ratio comprising removing T helper cells from a subject, contacting ex vivo the cells with an effective dose of an active substance selected from the group consisting of o) IL-2 and SDF-1α, p) a stimulant of IL-2 and a stimulant of SDF-1α, q) an antagonist to IL-4 and an antagonist to SDF-1α, r) an inhibitor of ZAP-70 or NFAT2, s) a stimulant of Syk or NFAT1, t) an IL-2 stimulating adjuvant and SDF-1α, u) a functional derivative, analogue or part of any of the substances o)-t) or v) a combination of any of the substances o)-u).

43. A method of claim 41 comprising subjecting the subject or recipient to be treated to a second treatment involving the manipulation of the immune system.

44. A method according to claim 43, wherein the second treatment involving the manipulation of the immune system is selected from the group consisting of a vaccination, antigen specific immunotherapy, allergen specific immunotherapy, nonspecific immunotherapy and an organ transplantation.

45. An antisense peptide nucleic acid (PNA) that is complementary to a DNA molecule encoding the tyrosine kinase Syk or a part thereof.

46. A PNA according to claim 45 comprising 5-25 bases.

47. A PNA according to claim 45 having the sequence of SEQ ID NO. 01.

48. An antisense peptide nucleic acid (PNA) that is complementary to a DNA molecule encoding the tyrosine kinase Syk or a part thereof for preventing or treating a Th1 cell-related disease by reducing the Th1/Th2 ratio.

49. A method of preventing or treating a Th1 cell-related disease by reducing the Th1/Th2 ratio comprising administering to a subject an effective dose of an antisense peptide nucleic acid (PNA) that is complementary to a DNA molecule encoding the tyrosine kinase Syk or a part thereof.

50. An antisense peptide nucleic acid (PNA) that is complementary to a DNA molecule encoding the tyrosine kinase ZAP-70 or a part thereof.

51. A PNA according to claim 50 comprising 5-25 bases.

52. A PNA according to claim 50 having the sequence of SEQ ID NO. 02.

53. An antisense peptide nucleic acid (PNA) that is complementary to a DNA molecule encoding the tyrosine kinase ZAP-70 or a part thereof for preventing or treating a Th2 cell-related disease by increasing the Th1/Th2 ratio.

54. A method of preventing or treating a Th2 cell-related disease by increasing the Th1/Th2 ratio comprising administering to a subject an effective dose of an antisense peptide nucleic acid (PNA) that is complementary to a DNA molecule encoding the tyrosine kinase ZAP-70 or a part thereof.

55. An in vitro diagnostic method of evaluating the T helper cell profile of a subject comprising obtaining a T helper cell containing sample from the subject, measuring the level of phosphorylated Syk, phosphorylated ZAP-70, intranucleic NFAT1 and/or intranucleic NFAT2 in the sample and using the measuring results obtained to assess the Th1/Th2 level.

56. An in vitro method of testing the effect of a product or a method on the Th1/Th2 ratio, comprising obtaining a T helper cell containing culture with a known Th1/Th2 ratio, subjecting the T helper cells to the product or method, measuring the level of phosphorylated Syk, phosphorylated ZAP-70, intranucleic NFAT1 and/or intranucleic NFAT2 in the sample and using the measuring results obtained to assess the change in Th1/Th2 level.

57. A diagnostic test kit comprising one or more probes specific for binding to phosphorylated Syk, phosphorylated ZAP-70, intranucleic NFAT1 and/or intranucleic NFAT2, and optionally a detection system.

58. A method of producing a culture enriched in Th1 cells comprising obtaining a T helper cell containing sample, subjecting the sample to an active substance selected from the group consisting of a) IL-2 and SDF-1α, b) a stimulant of IL-2 and a stimulant of SDF-1α, c) an antagonist to IL-4 and an antagonist to SDF-1α, d) an inhibitor of ZAP-70 or NFAT2, e) a stimulant of Syk or NFAT1, f) a functional derivative, analogue or part of any of the substances a)-e) or g) a combination of any of the substances a)-f) to increase the Th1/Th2 ratio.

59. A method of producing a culture enriched in Th2 cells comprising obtaining a T helper cell containing sample, subjecting the sample to an active substance selected from the group consisting of a) IL-4 and SDF-1α, b) a stimulant of IL-4 and a stimulant of SDF-1α, c) an antagonist to IL-2 and an antagonist to SDF-1α, d) an inhibitor of Syk or NFAT1, e) a stimulant of ZAP-70 or NFAT2, f) a functional derivative, analogue or part of any of the substances a)-e) or g) a combination of any of the substances a)-f) to decrease the Th1/Th2 ratio.

60. A method of claim 21 comprising subjecting the subject or recipient to be treated to a second treatment involving the manipulation of the immune system.

61. A method of claim 42 comprising subjecting the subject or recipient to be treated to a second treatment involving the manipulation of the immune system.

62. A PNA according to claim 45 comprising 10-20 bases.

63. A PNA according to claim 45 comprising 13-18 bases.

64. A PNA according to claim 46 having the sequence of SEQ ID NO. 01.

65. A PNA according to claim 50 comprising 10-20 bases.

66. A PNA according to claim 50 comprising 13-18 bases.

67. A PNA according to claim 51 having the sequence of SEQ ID NO. 02.
